SEQUENCE LISTING (18 sequences)

SEQ ID NO: 1 - DNA, 16 bp, Artificial Sequence
  Description of Artificial Sequence Synthetic oligonucleotide
agcgaccaccaacacg

SEQ ID NO: 2 - DNA, 17 bp, Artificial Sequence
  Description of Artificial Sequence Synthetic oligonucleotide
aagcgaccaccaacaca

SEQ ID NO: 3 - DNA, 25 bp, Artificial Sequence
  Description of Artificial Sequence Synthetic oligonucleotide
aaaagtgctcatctgtgaactctat

SEQ ID NO: 4 - DNA, 25 bp, Artificial Sequence
  Description of Artificial Sequence Synthetic oligonucleotide
aaaagtgctcatctgtgaactctac

SEQ ID NO: 5 - DNA, 23 bp, Artificial Sequence
  Description of Artificial Sequence Synthetic oligonucleotide
caaaggcctagaggagagattac

SEQ ID NO: 6 - DNA, 23 bp, Artificial Sequence
  Description of Artificial Sequence Synthetic oligonucleotide
caaaggcctagaggagagattat

SEQ ID NO: 7 - DNA, 23 bp, Artificial Sequence
  Description of Artificial Sequence Synthetic oligonucleotide
gagtatctctgctctagacctcg

SEQ ID NO: 8 - DNA, 23 bp, Artificial Sequence
  Description of Artificial Sequence Synthetic oligonucleotide
gagtatctctgctctagacctca

SEQ ID NO: 9 - DNA, 24 bp, Artificial Sequence
  Description of Artificial Sequence Synthetic oligonucleotide
cagcatctgagcattagtctttaa

SEQ ID NO: 10 - DNA, 25 bp, Artificial Sequence
  Description of Artificial Sequence Synthetic oligonucleotide
acagcatctgagcattagtctttac

SEQ ID NO: 11 - DNA, 26 bp, Artificial Sequence
  Description of Artificial Sequence Synthetic oligonucleotide
catgacaagtctctgaataagaagtc

SEQ ID NO: 12 - DNA, 26 bp, Artificial Sequence
  Description of Artificial Sequence Synthetic oligonucleotide
catgacaagtctctgaataagaagtg

SEQ ID NO: 13 - DNA, 14 bp, Artificial Sequence
  Description of Artificial Sequence Synthetic oligonucleotide
tacactgccaggca

SEQ ID NO: 14 - DNA, 24 bp, Artificial Sequence
  Description of Artificial Sequence Synthetic oligonucleotide
tttttttttttttttttttttttt

SEQ ID NO: 15 - DNA, 90 bp, Artificial Sequence
  Description of Artificial Sequence Synthetic oligonucleotide
ccccccccccccaaaaaaaaaaaaaaaaaaaaaaaaaaaaaaaaaaaaaaaaaaaaaaaaaaacacagctgaggataggacataataagc

SEQ ID NO: 16 - DNA, 80 bp, Artificial Sequence
  Description of Artificial Sequence Synthetic oligonucleotide
ctcagctgtgtaacaacatgaagattgtaggtcagaactcacctgttagaaactgtgaagatcgcttattatgtcctatc

SEQ ID NO: 17 - DNA, 18 bp, Artificial Sequence
  Description of Artificial Sequence Synthetic oligonucleotide
tgtcctatcctcagctgg

SEQ ID NO: 18 - DNA, 22 bp, Artificial Sequence
  Description of Artificial Sequence Synthetic oligonucleotide
cctacaatcttcatgttgttac

(19) United States
(12) Patent Application Publication
(10) Pub. No.: US 2004/0014078 A1
(43) Pub. Date: Jan. 22, 2004

(54) Compositions and methods for rolling circle amplification

(76) Inventors: James Xia, Chandler, AZ (US); Charles K. Brush, Whitefish Bay, WI (US); Vineet Gupta, Reading, MA (US); Heshu Huang, Cherry Hill, NJ (US); Changming Li, Palatine, IL (US); George Maracas, Phoenix, AZ (US); Robert Marrero, Pasadena, CA (US); Melissa L. Ray, Chandler, AZ (US); Lei Sun, Santa Clara, CA (US); Peiming Zhang, Gilbert, AZ (US)

Correspondence Address: Amersham Biosciences Corp., 800 Centennial Avenue, Piscataway, NJ, 08855, US

(21) Appl. No.: 10/360,511

(22) Filed: Feb. 6, 2003

Related U.S. Application Data
(60) Provisional application No. 60/355,374, filed on Feb. 6, 2002.

(51) Int. Cl. C12Q 1/68; C12P 19/34

(52) U.S. Cl. 435/6; 435/91.2

(57) ABSTRACT

This invention is directed to novel methods of amplifying and detecting DNA. More specifically, the invention applies variations of Rolling Circle Amplification to several detection platforms.

### CROSS REFERENCE TO RELATED APPLICATIONS

This application claims the benefit of the filing date of U.S. Ser. No. 60/355,374, filed Feb. 6, 2002.

### FIELD OF THE INVENTION

The invention is directed to novel methods of amplifying and detecting DNA. More specifically, the invention applies variations of Rolling Circle Amplification to several detection platforms.

### BACKGROUND OF THE INVENTION

The detection of specific nucleic acids is an important tool for diagnostic medicine and molecular biology research. Gene probe assays currently play roles in identifying infectious organisms such as bacteria and viruses, in probing the expression of normal genes and identifying mutant genes such as oncogenes, in typing tissue for compatibility preceding tissue transplantation, in matching tissue or blood samples for forensic medicine, and for exploring homology among genes from different species.

Ideally, a gene probe assay should be sensitive, specific and easily automatable (for a review, see Nickerson, Current Opinion in Biotechnology 4:48-51 (1993)). The requirement for sensitivity (i.e. low detection limits) has been greatly alleviated by the development of the polymerase chain reaction (PCR) and other amplification technologies which allow researchers to amplify exponentially a specific nucleic acid sequence before analysis (for a review, see Abramson et al., Current Opinion in Biotechnology, 4:41-47 (1993)).

Sensitivity, i.e. detection limits, remain a significant obstacle in nucleic acid detection systems, and a variety of techniques have been developed to address this issue. Briefly, these techniques can be classified as either target amplification or signal amplification. Target amplification involves the amplification (i.e. replication) of the target sequence to be detected, resulting in a significant increase in the number of target molecules. Target amplification strategies include the polymerase chain reaction (PCR), strand displacement amplification (SDA), and nucleic acid sequence based amplification (NASBA).

Alternatively, rather than amplify the target, alternate techniques use the target as a template to replicate a signaling probe, allowing a small number of target molecules to result in a large number of signaling probes, that then can be detected. Signal amplification strategies include the ligase chain reaction (LCR), cycling probe technology (CPT), Invader, Q-beta replicase (QBR), and the use of “amplification probes” such as “branched DNA” that result in multiple label probes binding to a single target sequence.

Of particular interest herein is rolling circle amplification (RCA). RCA is an isothermal process for generating multiple copies of a sequence. In rolling circle DNA replication in vivo, a DNA polymerase extends a primer on a circular template (Kornberg, A. and Baker, T. A. DNA Replication, W. H. Freeman, New York, 1991). The product consists of tandemly linked copies of the complementary sequence of the template. RCA is a method that has been adapted for use in vitro for DNA amplification (Fire, A. and Si-Qun Xu, Proc. Natl. Acad Sci. USA, 1995, 92:4641-4645; Lui, D., et al., J. Am. Chem. Soc., 1996,118:1587-1594; Lizardi, P. M., et al., Nature Genetics, 1998, 19:225-232; U.S. Pat. No. 5,714,320 to Kool). RCA can also be used in a detection method using a probe called a “padlock probe” (WO Pat. Ap. Pub. 95/22623 to Landegren; Nilsson, M., et al. Nature Genetics, 1997, 16:252-255, and Nilsson, M., and Landegren, U., in Landegren, U., ed., Laboratory Protocols for Mutation Detection, Oxford University Press, Oxford, 1996, pp. 135-138). DNA synthesis has been limited to rates ranging between 50 and 300 nucleotides per second (Lizardi, cited above and Lee, J., et al., Molecular Cell, 1998, 1:1001-1010).

Accordingly, it is an object of the present invention to provide a variety of improvements and novel configurations of RCA with subsequent detection on biochips.

### BRIEF DESCRIPTION OF THE DRAWINGS

FIG. 1A. Schematic representation of SBE-RCA. 5′-immobilized Single Base Extension (SBE) probes contains allele discriminating nucleotides at the 3′ terminus. During SBE, a single nucleotide is incorporated by DNA polymerase-mediated extension in the presence of a mixture of chain terminating, biotin-acyclo-nucleoside triphosphates, and hybridizing target. SBE signals are amplified by Rolling Circle Amplification (RCA). During RCA, Neutravidin (or α-biotin antibody) conjugated to an RCA primer binds to the biotin on the extended SBE probes. Rolling circle amplification is performed and the product is detected by hybridization with a fluorophore-labeled oligonucleotide “decorator”. The fluorescence signals are detected by scanning in a laser scanner and quantitated using the CodeLink software. Hyb signals were detected by hybridizing decorators either directly to the primer associated with the conjugate or after hybridization of RCA circle in the absence of RCA signal amplification.

FIG. 1B. RCA signal amplification of biotin-tagged oligonucleotides immobilized on HYDROGEL substrates. Microarrays containing a dilution series of immobilized biotin-tagged oligonucleotides were incubated with α-biotin-primer1 conjugate. Following RCA amplification, the product was detected directly by Hyb (upper panel) or RCA-mediated signal amplification (lower panel).

FIG. 1C. Quantitation of fluorescence spots SBE-RCA signals obtained from array in Fig 1b. Spots were quantified using the QuantArray Image software package. Average pixel intensity at each spot was plotted against the concentration of probe at the time of deposition on to microarrays—RCA (dotted line), Hyb (solid line). Insert depicts expanded region of probe concentrations from 0.1-1 nM, and includes the assays limit of detection (770 pM)

FIG. 2A. SNP genotyping with PCR targets. SBE reactions included 1 ng of 906 and 1.5 ng of LPL2 PCR amplified targets. Map shows location of SBE primers on the array. R—Represented allele; U: Non-presented allele; APOE—Self-extending primer control for SBE; POS1 & POS2—Positive controls for RCA, M: Cy5 labeled marker oligonucleotide. Heterozygotes: 906 and 198; Homozygotes: 750, 2068, 1820, and LPL2.

FIGS. 2B and 2C. Plots of SBE-RCA signals over a range of 906 (heterozygote) and LPL2 (homozygote) target concentrations. Mean signal intensity of SBE-RCA ( ) and SBE-Hyb were plotted against amount of synthetic target used in the SBE reaction.

FIG. 2D. Allele Discrimination (AD) with SBE RCAT over a range of target concentrations. Filled squares: LPL2 (Homozygote); Hollow triangles: 906 (Heterozygote).

FIG. 3A. Signal-to-noise ratio vs. SBE cycle number. SBE and RCA performed as described in Experimental protocol. Assays employed 5 ng of each target amplicon.

FIG. 3B. Effect of target input on SBE-RCA signal-to-noise ratio. Assays were performed as described in Experimental protocol, with target input ranging form 0.5 to 20 ng per 80 μl assay. Heterozygous target: 906, homozygous targets: 750, LPL2.

FIG. 3C. Allele discrimination vs. SBE cycle number. SBE and RCA performed as described in Experimental protocol. Assays employed 5 ng of each target amplicon

FIG. 3D. Effect of target input on SBE-RCA allele discrimination ratio. Assays were performed as described in Experimental protocol, with target input ranging form 0.5 to 20 ng per 80 μl assay. Heterozygous target: 906, homozygous targets: 750, LPL2.

FIG. 4A. Geno Chip: RCA signal amplification with unmodified template. Microarray image of SBE-RCA with primers for human repetitive sequence families. The two spots in each column are duplicates. SBE probes (with haploid genome copy numbers in parentheses) deposited in each column are 1- SMR4.T.S (10⁶); 3-ALR87.C (5×10⁵); 5-ALR259.G (5×10⁵); 7-ALR86.G (5×10⁵); 9-MER5.C (5×10⁴); 11-L1TR.C (5×10⁴); 13-MAR.T (10⁴); 15-MER28.8.8.T2.G (10⁴); 17-MER6.T (10³); 19-MAR2.C (10³). Columns with even numbers contained the corresponding mismatched primers for each of the above primers. The SBE reaction contained 0.5 ug of sonicated human genomic DNA.

FIG. 4B. Mean GENO-1 signal intensities. Quantified signals from 4a plotted (background subtracted using ‘no target’ controls).

FIG. 4C. Numeric amplification and allele ratio factors on repetitive markers.

FIG. 5. SNP targets and Hydrogel-immobilized SBE-probe oligonucleotides used in this study. Nomenclature: wiaf-198 (target locus); C (Polymorphic base call); A (Antisense strand); or S (Sense strand).; Coriell Cell Repositories sample set M08PDR, PD007.

FIG. 6. Characteristics of SBE signals amplified by RCA. Heterozygous targets: 906 and 198; homozygous targets: 750, 1820, 2068 and LPL2. See FIG. 5 for represented alleles. Data are form two experiments with target input levels between 4-12 ng of amplicon target per assay, employing 2 SBE cycles (See Experimental protocols for details).

FIGS. 7A-7F depict one embodiment where the capture probe (11) is attached to a substrate (10) at both its termini. The capture probe comprises a first domain (12). This first domain is substantially complimentary to a domain of an open circle probe (13). That is, the open circle probe (13) comprises a domain which is substantially complimentary to a target sequence.(14). The target sequence (14) hybridizes to the open circle probe (13) to form a first hybridization complex (20). The first hybridization complex (20) is contacted with ligase to form a second hybridization complex (21). The capture probe (11) is then contacted with a cleavage agent to cleave the probe and allow for Rolling Circle Amplification to proceed. Extension enzyme and NTPs are added to the second hybridization complex (21) to form an extended capture probe(22). A fluorescent dye (15) is added, generally in the form of either a label probe or direct incorporation into the extended probe, to the extended capture probe (22) and the extended capture probe (22) is detected.

FIG. 8 depicts one embodiment of the invention where detection if the extended capture probe (22) is detected via e-detection. In this embodiment a capacitor (30) is used to measure the dielectric change after extension of the capture probe (22).

FIG. 9 depicts one embodiment of the invention where detection of the extended capture probe (22) is detected via e-Sensor™. In this embodiment a gold electrode is the substrate (40), and is covered is Self-Assembeled Monolayers (SAMs) (42). Also, the extended capture probe (22) is electrochemically labeled with an electron transfer moiety (ETM) (41). The electrons flow from the ETM (41) to the electrode (40) and back. This creates a detectable signal.

FIGS. 10A-10C depict one embodiment of the invention where a target sequence (14) comprises a first target domain (34) and a second target domain (35). A device comprises a substrate(10), which comprises a capture probe (11) that is substantially complementary to a first domain (34) of said target sequence (14). The capture probe (11) is then contacted with the target sequence (14); and a rolling circle primer (44). The rolling circle primer (44) comprises a first domain (46) that is substantially complementary to the second domain of said target sequence (34); and a second domain (45) substantially complementary to a first domain of a circularized probe (47). This contact forms a first hybridization complex (20). The first hybridization complex (20) is contacted with a ligase such that capture probe (11) and the rolling circle primer (44) ligate. Next, the second domain of the rolling circle primer (45) is hybridized to a circularized probe (47) to form a second hybridization complex (21). An extension enzyme and NTPs are added to the second hybridization complex (21) to form an extended capture probe (22) and the extended capture probe (22) is detected.

FIG. 11 depicts one embodiment of the invention where the extended capture probe (22) is detected via a capacitor (30). The capacitor (30) is used to measure the dielectric change after extension of the capture probe (22).

FIG. 12 depicts one embodiment of the invention where a measurement of electrical current genetrated following oxidation of guanine in the presence of a soluble, redox-active mediator (e.g. ruthenium tris(2,2′-bipyridine)). The first hybridization complex (20) is oxidized and the more oxidized guanines, the stronger the signal. The electrons flow first hybridization complex (20) to the electrode (40) and back. This creates a detectable signal.

FIG. 13 depicts one embodiment of the invention where detection of the extended capture probe (22) is detected via e-Sensor™. In this embodiment a gold electrode is the substrate (40), and is covered is Self-Assembeled Monolayers (SAMs) (42). Also, the extended capture probe (22) is electrochemically labeled with an ETM (41). The electrons flow from the ETM (41) to the electrode (40) and back. This creates a detectable signal.

FIG. 14A depicts one embodiment where SNP genotyping is performed using CodeLink™. Wherein a hybridization complex (20) comprising a target sequence (14) and a capture probe (11) with an interrogation position (53) is contacted with a hapten labeled nucleotide (55). Only under conditions wherein a secondary probe (56) comprising the binding partner of the hapten (56) is perfectly complementary, do the hybridization complex (20) and secondary probe (56) hybridize. The secondary probe (56) comprises a fluorescent dye or ETM (41) which is detected.

FIG. 14B depicts another embodiment where SNP genotyping is performed using CodeLink™. Wherein a hybridization complex (20) comprising a target sequence (14) and a capture probe (11) with an interrogation position (53) is contacted with a hapten labeled nucleotide (55). Only under conditions wherein a secondary probe (56) comprising the binding partner of the hapten (56) is perfectly complementary, do the hybridization complex (20) and secondary probe (56) hybridize. A closed circle probe (47) comprising a rolling circle priming sequence is added with an extension enzyme and NTPs to extend the primer. ETM (41) are added to hybridize with the extended primer (58). The ETMs are then detected.

FIG. 14C depicts another embodiment where SNP genotyping is performed using CodeLink™. Wherein a closed circle probe (47) is added to a hybridization complex. No extention takes place, only detection.

FIG. 15 depicts an alternate scheme of the invention.

FIG. 16 depicts an alternate scheme of the invention.

FIG. 17 depicts an alternate scheme of the invention.

FIG. 18 depicts an alternate scheme of the invention.

FIG. 19 depicts an alternate scheme of the invention.

### DETAILED DESCRIPTION OF THE INVENTION

The present invention is generally directed to the detection, genotyping and/or quantification of target sequences in a sample using a variety of novel configurations of Rolling Circle Amplification (“RCA”).

One aspect of the invention is directed to a method of detecting the presence of a target sequence using a capture probe. The capture probe consists of two different domains. The first domain is substantially complementary to a open circle probe, and the second domain contains a cleavage site. The capture probe is attached to the substrate at both of its termini to form an “arch” shape.

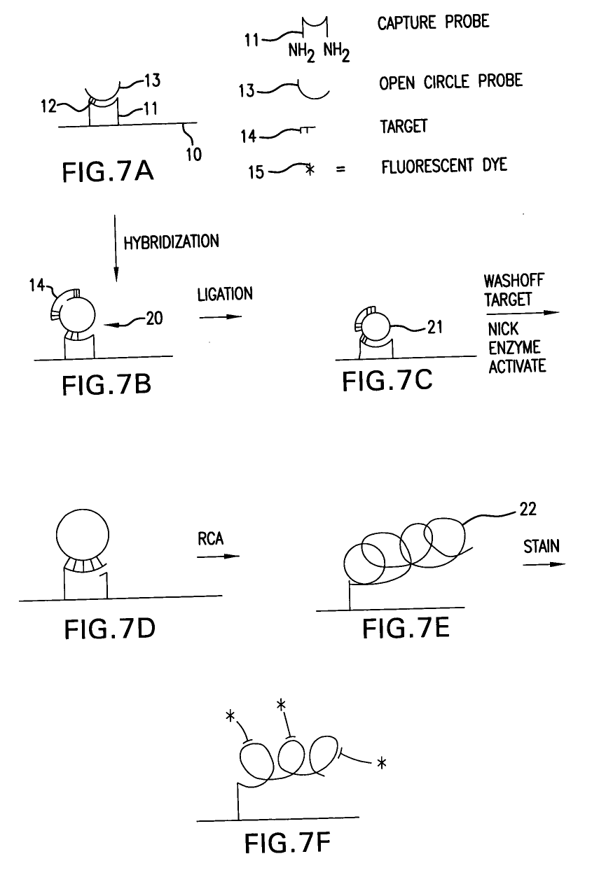

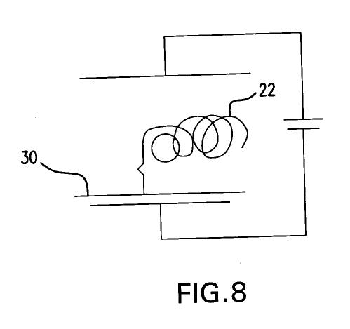

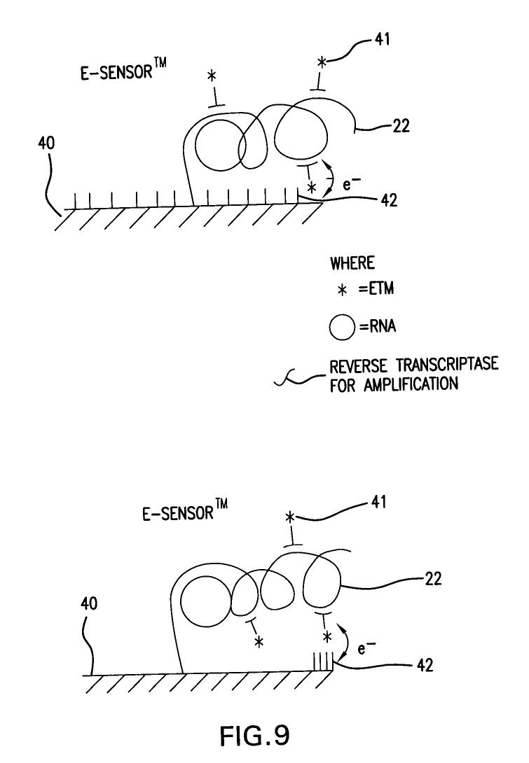

First, the capture probe is contacted with the target sequence and the open circle probe to form a hybridization complex. Next, the hybridization complex is treated with a ligase such that the open circle probe circularizes to form a distinct second hybridization complex. The capture probe is then treated with a cleavage agent to cleave the probe. Next, Rolling Circle Amplification is performed and an extended capture probe is formed. Finally, the extended capture probe is detected. This is generally depicted in FIGS. 7A-7F.

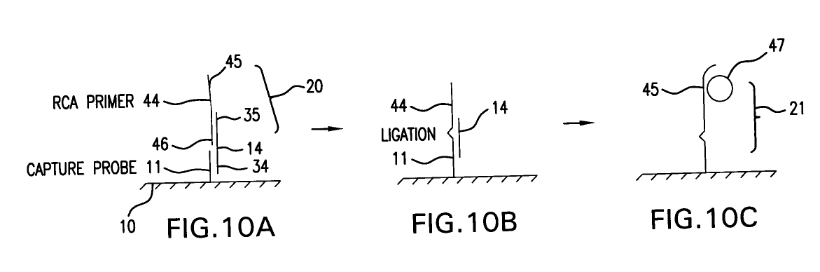

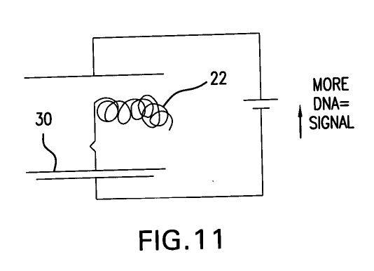

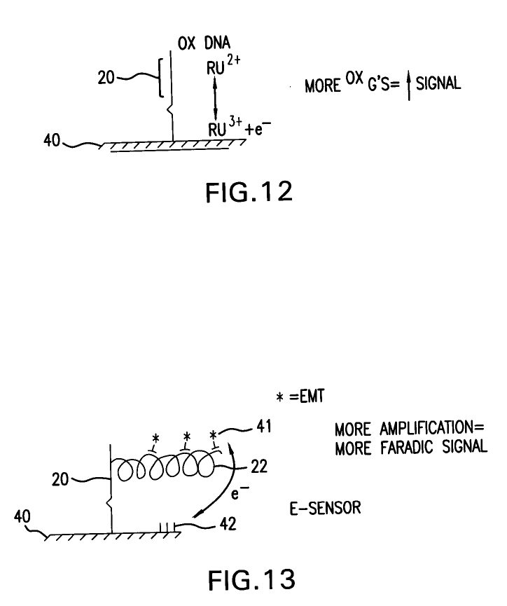

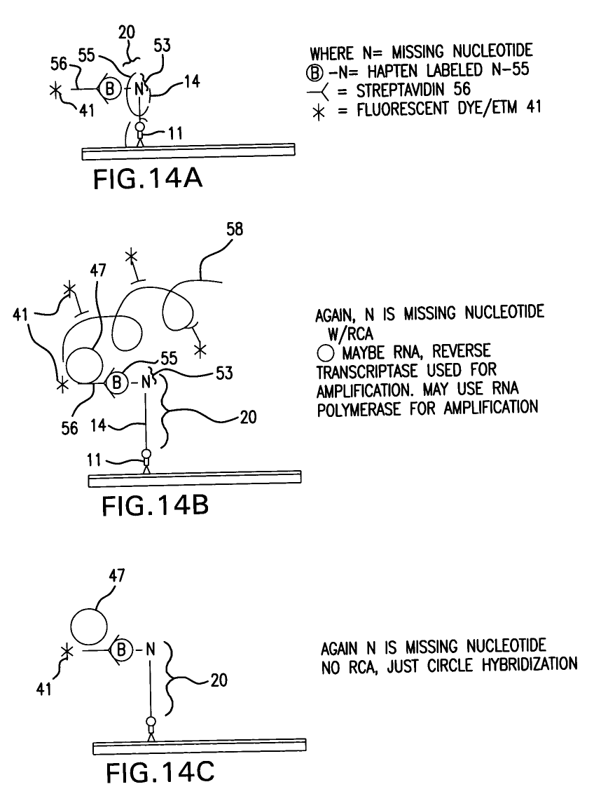

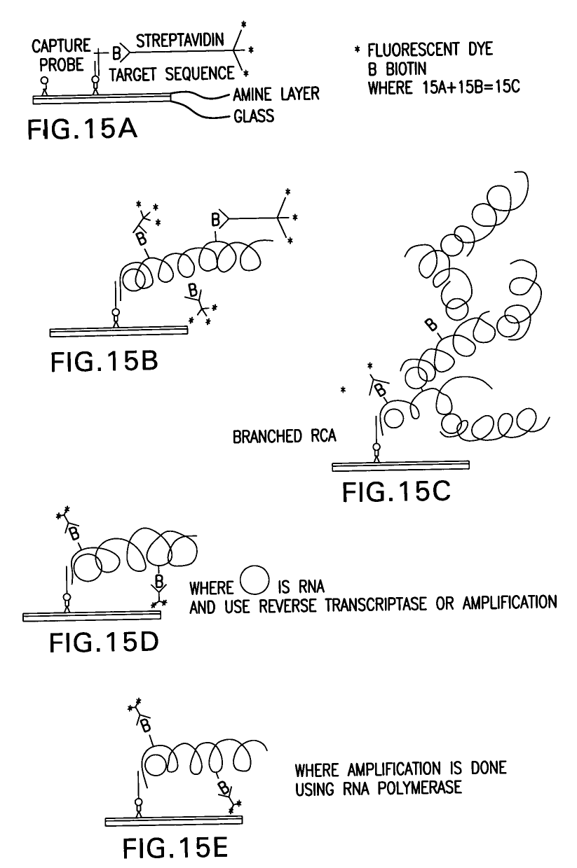

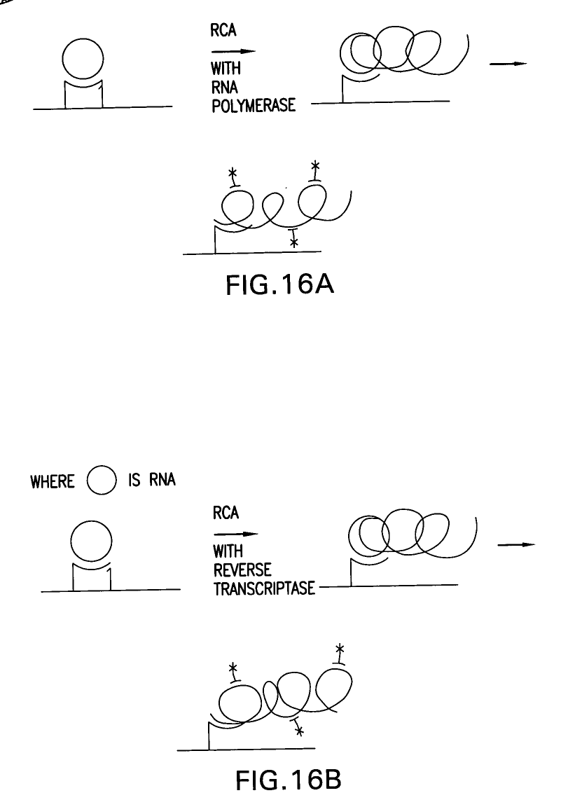

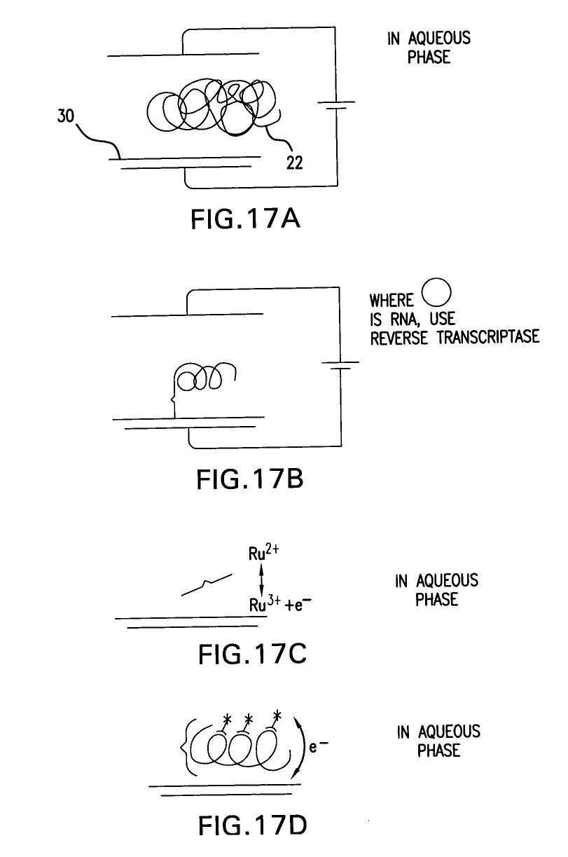

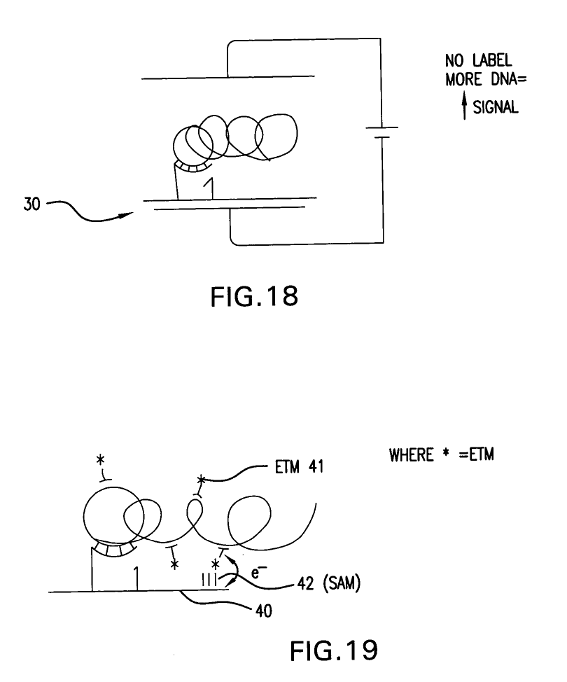

Another aspect of the invention, depicted in FIGS. 10A-10C, is directed to detecting the presence of a target sequence, having two distinct domains, using a capture probe that is substantially complementary to a first domain of the target sequence. The capture probe may either be attached to a substrate (solid phase) or it may be in solution phase. The capture probe is contacted with the target sequence and a rolling circle primer comprising two domains. The first domain is substantially complementary to the second domain of the target sequence and the second domain of the primer is substantially complementary to a circularized probe. When in contact with one another, the primer, target and circularized probe form a hybridization complex. The hybridization complex is then treated with a ligase so that capture probe and said rolling circle primer ligate. Next, the second domain of the rolling circle primer is hybridized to the circularized probe to form a second hybridization complex. Finally RCA is performed and the extended capture probe is detected.

Yet another aspect of the invention is directed to detecting the presence of a target sequence having first and second target domains adjacent to one another. The second target domain and a capture probe are brought together with a ligation probe. The ligation probe contains two domains. The first domain is substantially complementary to the second domain of the target sequence, and the second to a rolling circle primer. When the nucleotides at the adjacent termini of the capture probe and the ligation probe are perfectly complementary to the respective target nucleotides, the capture probe and the ligation probe are ligated to form a ligated probe. The rolling circle primer of the ligated probe is then hybirdized with a rolling circle priming sequence of a closed circle probe to form a rolling circle hybridization structure. RCA is then performed and the extended product is then detected.

Accordingly, the present invention is directed to the detection, genotyping and/or quantification of target sequences in a sample using a variety of configurations of Rolling Circle Amplification (“RCA”).

As will be appreciated by those in the art, the sample solution may comprise any number of things, including, but not limited to, bodily fluids (including, but not limited to, blood, urine, serum, lymph, saliva, anal and vaginal secretions, perspiration and semen) or solid tissue samples, of virtually any organism, with mammalian samples being preferred and human samples being particularly preferred); environmental samples (including, but not limited to, air, agricultural, water and soil samples); biological warfare agent samples; research samples; purified samples, such as purified or raw genomic DNA, RNA, proteins, etc.; raw samples (bacteria, virus, genomic DNA, mRNA, etc.). As will be appreciated by those in the art, virtually any experimental manipulation may have been done on the sample.

There is no limitation as to the source of the template nucleic acid: it can be from a eukaryote, e.g., from a mammal, such as human, mouse, ovine, bovine, or from a plant; it can be from a prokaryote, e.g., bacteria, protozoan; and it can also be from a virus.

Nucleic acid specimens may be obtained from an individual of the species that is to be analyzed using either “invasive” or “non-invasive” sampling means. A sampling means is said to be “invasive” if it involves the collection of nucleic acids from within the skin or organs of an animal (including, especially, a murine, a human, an ovine, an equine, a bovine, a porcine, a canine, or a feline animal). Examples of invasive methods include blood collection, semen collection, needle biopsy, pleural aspiration, umbilical cord biopsy, etc. Examples of such methods are discussed by Kim, C. H. et al. (J. Virol. 66:3879-3882 (1992)); Biswas, B. et al. (Annals NY Acad. Sci. 590:582-583 (1990)); Biswas, B. et al. (J. Clin. Microbiol. 29:2228-2233 (1991)).

In contrast, a “non-invasive” sampling means is one in which the nucleic acid molecules are recovered from an internal or external surface of the animal. Examples of such “non-invasive” sampling means include “swabbing,” collection of tears, saliva, urine, fecal material, sweat or perspiration, hair etc. As used herein, “swabbing” denotes contacting an applicator/collector (“swab”) containing or comprising an adsorbent material to a surface in a manner sufficient to collect live cells, surface debris and/or dead or sloughed off cells or cellular debris. Such collection may be accomplished by swabbing nasal, oral, rectal, vaginal or aural orifices, by contacting the skin or tear ducts, by collecting hair follicles, etc.

Methods for isolating nucleic acid specimens are known in the art, and will depend on the type of nucleic acid isolated. When the nucleic acid is RNA, care to avoid RNA degradation must be taken, e.g., by inclusion of RNAsin. For example, genomic DNA can be prepared from human cells as described, e.g., in U.S. Pat. No. 6,027,889; incorporated herein by reference in its entirety.

The present invention provides compositions and methods for genotyping and/or detecting the presence or absence of target nucleic acid sequences in a sample. By “nucleic acid” or “oligonucleotide” or grammatical equivalents herein means at least two nucleotides covalently linked together. A nucleic acid of the present invention will generally contain phosphodiester bonds, although in some cases, as outlined below, such as in the design of probes, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, A Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of labels, or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine, hypoxathanine, isocytosine, isoguanine, etc. A preferred embodiment utilizes nucleic acid probes comprising some proportion of uracil, as is more fully outlined below. One embodiment utilizes isocytosine and isoguanine in nucleic acids designed to be complementary to other probes, rather than target sequences, as this reduces non-specific hybridization, as is generally described in U.S. Pat. No. 5,681,702. As used herein, the term “nucleoside” includes nucleotides as well as nucleoside and nucleotide analogs, and modified nucleosides such as labeled nucleosides. In addition, “nucleoside” includes non-naturally occuring analog structures. Thus for example the individual units of a peptide nucleic acid, each containing a base, are referred to herein as a nucleoside. Similarly, the term “nucleotide” (sometimes abbreviated herein as “NTP”), includes both ribonucleic acid and deoxyribonucleic acid (sometimes abbreviated herein as “dNTP”). While many descriptions below utilize the term “dNTP”, it should be noted that in many instances NTPs may be substituted, depending on the template and the enzyme.

In another preferred embodiment, terminal transferase can be used to add nucleotides comprising separation labels such as biotin to any linear molecules, and then the mixture run through a strepavidin system to remove any linear nucleic acids, leaving only the closed circular probes. For example, when genomic DNA is used as the target, this may be biotinylated using a variety of techniques, and the precircle probes added and circularized. Since the circularized probes are catenated on the genomic DNA, the linear unreacted precircle probes can be washed away. The closed circle probes can then be cleaved, such that they are removed from the genomic DNA, collected and amplified. Similarly, terminal transferase may be used to add chain terminating nucleotides, to prevent extension and/or amplification. Suitable chain terminating nucleotides include, but are not limited to, dideoxy-triphosphate nucleotides (ddNTPs), halogenated dNTPs and acyclo nucleotides (NEN). These latter chain terminating nucleotide analogs are particularly good substrates for Deep vent (exo⁻) and thermosequenase.

The compositions and methods of the invention are directed to the detection of target sequences. The term “target sequence” or “target nucleic acid” or grammatical equivalents herein means a nucleic acid sequence on a single strand of nucleic acid. The target sequence may be a portion of a gene, a regulatory sequence, genomic DNA, cDNA, RNA including mRNA and rRNA, or others. As is outlined herein, the target sequence may be a target sequence from a sample, or a secondary target such as a product of a genotyping or amplification reaction such as a ligated circularized probe, an amplicon from an amplification reaction such as PCR, etc. Thus, for example, a target sequence from a sample is amplified to produce a secondary target (amplicon) that is detected. Alternatively, as outlined more fully below, what may be amplified is the probe sequence, although this is not generally preferred. The target sequence may be any length, with the understanding that longer sequences are more specific. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others. As is outlined more fully below, probes are made to hybridize to target sequences to determine the presence, sequence or quantity of a target sequence in a sample. Generally speaking, this term will be understood by those skilled in the art. Preferred target sequences range from about 20 to about 1,000,000 in size, more preferably from about 50 to about 10,000, with from about 40 to about 50,000 being most preferred.

If required, the target sequence is prepared using known techniques. For example, the sample may be treated to lyse the cells, using known lysis buffers, sonication, electroporation, etc., with purification and amplification as outlined below occurring as needed, as will be appreciated by those in the art. In addition, the reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents which may be included in the assays. These include reagents like salts, buffers, neutral proteins, e.g. albumin, detergents, etc., which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target.

In addition, in most embodiments, double stranded target nucleic acids are denatured to render them single stranded so as to permit hybridization of the primers and other probes of the invention. A preferred embodiment utilizes a thermal step, generally by raising the temperature of the reaction to about 95° C., although pH changes and other techniques may also be used.

In addition, in some cases, for example when genomic DNA is to be used, it can be captured, such as through the use of precipitation or size exclusion techniques. Alternatively, DNA can be processed to yield uniform length fragments using techniques well known in the art, such as, e.g., hydrodynamic shearing or restriction endonucleases.

The target sequences of the present invention in many cases comprise at least a first and a second target domain. Target domains are portions of the target sequence. In general, each target domain may be any length, with the understanding that longer sequences are more specific. The proper length of the target domains in a probe will depend on factors including the GC content of the regions and their secondary structure. The considerations are similar to those used to identify an appropriate sequence for use as a primer, and are further described below. The length of the probe and GC content will determine the Tm of the hybrid, and thus the hybridization conditions necessary for obtaining specific hybridization of the probe to the template nucleic acid. These factors are well known to a person of skill in the art, and can also be tested in assays. An extensive guide to the hybridization of nucleic acids is found in Tijssen (1993), “Laboratory Techniques in biochemistry and molecular biology-hybridization with nucleic acid probes.” Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Highly stringent conditions are selected to be equal to the Tm point for a particular probe. Sometimes the term “Td” is used to define the temperature at which at least half of the probe dissociates from a perfectly matched target nucleic acid. In any case, a variety of estimation techniques for estimating the Tm or Td are available, and generally described in Tijssen, supra. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm and Td are available and appropriate in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the annealing of the probe to the template DNA.

The terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain.

The stability difference between a perfectly matched duplex and a mismatched duplex, particularly if the mismatch is only a single base, can be quite small, corresponding to a difference in Tm between the two of as little as 0.5 degrees. See Tibanyenda, N. et al., Eur. J. Biochem. 139:19 (1984) and Ebel, S. et al., Biochem. 31:12083 (1992). More importantly, it is understood that as the length of the homology region increases, the effect of a single base mismatch on overall duplex stability decreases. Thus, where there is a likelihood that there will be mismatches between the probe and the target domains, it may be advisable to include a longer targeting domain in the probe.

Thus, the specificity and selectivity of the probe can be adjusted by choosing proper lengths for the targeting domains and appropriate hybridization conditions. When the template nucleic acid is genomic DNA, e.g., mammalian genomic DNA, the selectivity of the targeting domains must be high enough to identify the correct base in 3×10⁹ in order to allow processing directly from genomic DNA. However, in situations in which a portion of the genomic DNA is isolated first from the rest of the DNA, e.g., by separating one or more chromosomes from the rest of the chromosomes, the selectivity or specificity of the probe is less important.

As outlined herein, the target domains may be adjacent (i.e. contiguous) or separated, i.e. by a “gap”. If separated, the target domains may be separated by a single nucleotide or a plurality of nucleotides, with from 1 to about 2000 being preferred, and from 1 to about 500 being especially preferred, although as will be appreciated by those in the art, longer gaps may find use in some embodiments.

In a preferred embodiment, e.g. for genotyping reactions, as is more fully outlined below, the target sequence comprises a position for which sequence information is desired, generally referred to herein as the “detection position”. In a particularly preferred embodiment, the detection position is a single nucleotide, although in alternative embodiments, it may comprise a plurality of nucleotides, either contiguous with each other or separated by one or more nucleotides. By “plurality” as used herein is meant at least two. As used herein, the base which base pairs with the detection position base in a target is termed the “interrogation position”. In the case where a single nucleotide gap is used, the NTP that has perfect complementarity to the detection position is called an “interrogation NTP”.

It should be noted in this context that “mismatch” is a relative term and meant to indicate a difference in the identity of a base at a particular position, termed the “detection position” herein, between two sequences. In general, sequences that differ from wild type sequences are referred to as mismatches. However, and particularly in the case of SNPS, what constitutes “wild type” may be difficult to determine as multiple alleles can be relatively frequently observed in the population, and thus “mismatch” in this context requires the artificial adoption of one sequence as a standard. Thus, for the purposes of this invention, sequences are referred to herein as “perfect match” and “mismatch”. “Mismatches” are also sometimes referred to as “allelic variants”. The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation. The term “allelic variant of a polymorphic region of a gene” refers to a region of a gene having one of several nucleotide sequences found in that region of the gene in other individuals of the same species.

In the cases of probes, complementarity need not be perfect; there may be any number of base pair mismatches that will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under the selected reaction conditions.

The present invention provides devices comprising substrates with capture probes. By “device” herein is meant a piece of equipment or a mechanism designed to perform a special function. More specifically, the special function is to detect, genotype and quantify target sequences in a sample. CodeLink™ (fluorescence detection), e-Sensor (electrochemical detection), and e-detection (non-label detection) are all detection platforms and will be described in further detail below.

The devices comprise substrates. By “substrate” or “solid support” or other grammatical equivalents herein is meant any material that can be modified to contain discrete individual sites appropriate for the attachment or association of capture probes and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals (particularly electrodes), inorganic glasses, plastics, optical fiber bundles, and a variety of other polymers. In general, the substrates allow optical detection and do not themselves appreciably fluoresce.

The substrate comprises an array of capture probes. Accordingly, the present invention provides array compositions comprising at least a first substrate with a surface comprising individual sites. By “array” or “biochip” herein is meant a plurality of nucleic acids in an array format; the size of the array will depend on the composition and end use of the array. Nucleic acids. arrays are known in the art, and can be classified in a number of ways; both ordered arrays (e.g. the ability to resolve chemistries at discrete sites), and random arrays (e.g. bead arrays) are included. Ordered arrays include, but are not limited to, those made using photolithography techniques (Affymetrix GeneChip), spotting techniques (Synteni and others), printing techniques (Hewlett Packard and Rosetta), electrode arrays, three dimensional gel or gel pad arrays, etc. Liquid arrays may also be used.

The construction and use of solid phase nucleic acid arrays to detect target nucleic acids is well described in the literature. See, Fodor et al. (1991) Science, 251: 767-777; Sheldon et al. (1993) Clinical Chemistry 39(4): 718-719; Kozal et al. (1996) Nature Medicine 2(7): 753-759 and Hubbell U.S. Pat. No. 5,571,639. See also, Pinkel et al. PCT/US95/16155 (WO 96/17958). In brief, a combinatorial strategy allows for the synthesis of arrays containing a large number of probes using a minimal number of synthetic steps. For instance, it is possible to synthesize and attach all possible DNA 8 mer oligonucleotides (48, or 65,536 possible combinations) using only 32 chemical synthetic steps. In general, VLSIPS TM procedures provide a method of producing 4n different oligonucleotide probes on an array using only 4n synthetic steps.

Light-directed combinatorial synthesis of oligonucleotide arrays on a glass surface is performed with automated phosphoramidite chemistry and chip masking techniques similar to photoresist technologies in the computer chip industry. Typically, a glass surface is derivatized with a saline reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface.

A 96 well automated multiplex oligonucleotide synthesizer (A.M.O.S.) has also been developed and is capable of making thousands of oligonucleotides (Lashkari et al. (1995) PNAS 93: 7912). Existing light-directed synthesis technology can generate high-density arrays containing over 65,000 oligonucleotides (Lipshutz et al. (1995) BioTech. 19: 442.

Combinatorial synthesis of different oligonucleotide analogues at different locations on the array is determined by the pattern of illumination during synthesis and the order of addition of coupling reagents. Monitoring of hybridization of target nucleic acids to the array is typically performed with fluorescence microscopes or laser scanning microscopes. In addition to being able to design, build and use probe arrays using available techniques, one of skill is also able to order custom-made arrays and array-reading devices from manufacturers specializing in array manufacture. For example, Affymetrix Corp., in Santa Clara, Calif. manufactures DNA VLSIP TM arrays.

It will be appreciated that oligonucleotide design is influenced by the intended application. For example, where several oligonucleotide -tag interactions are to be detected in a single assay, e.g., on a single DNA chip, it is desirable to have similar melting temperatures for all of the probes. Accordingly, the length of the probes are adjusted so that the melting temperatures for all of the probes on the array are closely similar (it will be appreciated that different lengths for different probes may be needed to achieve a particular Tm where different probes have different GC contents). Although melting temperature is a primary consideration in probe design, other factors are optionally used to further adjust probe construction, such as selecting against primer self-complementarity and the like. The “active” nature of the devices provide independent electronic control over all aspects of the hybridization reaction (or any other affinity reaction) occurring at each specific microlocation. These devices provide a new mechanism for affecting hybridization reactions which is called electronic stringency control (ESC). For DNA hybridization reactions which require different stringency conditions, ESC overcomes the inherent limitation of conventional array technologies. The active devices of this invention can electronically produce “different stringency conditions” at each microlocation. Thus, all hybridizations can be carried out optimally in the same bulk solution. These arrays are described in U.S. Pat. No. 6,051,380 by Sosnowski et al.

In a preferred embodiment CodeLink™ array technology is used, CodeLink™ technology provides an apparatus for performing high-capacity biological reactions on a biochip comprising a substrate having an array of biological binding sites. It provides a hybridization chamber having one or more arrays, preferably comprising arrays consisting of hydrophilic, 3-dimensional gel and most preferably comprising arrays consisting of 3-dimensional polyacrylamide gels, wherein nucleic acid hybridization is performed by reacting a biological sample containing a target molecule of interest with a complementary oligonucleotide probe immobilized on the gel. Nucleic acid hybridization assays are advantageously performed using probe array technology, which utilizes binding of target single-stranded DNA onto immobilized oligonucleotide probes. Preferred arrays include those outlined in U.S. Ser. Nos. 09/458,501, 09/459,685, 09/464,490, 09/605,766, PCT/US00/34145, 09/492,013, PCT/US01 /02664, WO 01/54814, 09/458, 533, 09/344,217, PCT/US99/27783, 09/439,889, PCT/US00/42053 and WO 01/34292 all of which are hereby incorporated by reference in their entirety.

In another perferred embodiment eSensor™ array technology is used. eSensor™ technology uses self-assembled monolayers (SAMs) on surfaces for binding and detection of biological molecules. SAMs are alkyl chains that protect an electrode from solution electronically active agents (e.g. salts). Electrochemical labels (e.g. ferrocene), which are initially bound to the label probe, flow to the electrode and back producing a detectable signal. See for example WO98/20162; PCT US98/12430; PCT US98/12082; PCT US99/01705; PCT/US99/21683; PCT/US99/10104; PCT/US99/01703; PCT/US00/31233; U.S. Pat. Nos. 5,620,850; 6,197,515; 6,013,459; 6,013,170; and 6,065,573; and references cited therein.

In yet another preferred embodiment Xanthon™ array technology is used. Xanthon™ technology is an electrochemical platform that directly detects target nucleic acids without the need for sample purification, amplification or the use of fluorescent, chemiluminescent or radioactive labels. This technology relies on soluble electron transfer mediators to quantitate the number of oxidizable quanine residues on a surface. That is, when a target sequence is present, the amount of guanines increases, thus resulting in an increase of electron transfer. (See e.g. An Ionic Liquid Form of DNA: Redox-Active Molten Salts of Nucleic Acids. A. M. Leone, S. C. Weatherly, M. E. Williams, R. W. Murray, H. H. Thorp J. Am. Chem. Soc., 2001, 123, 218-222. Mediated electrochemical detection of nucleic acids for drug discovery and clinical diagnostics. N. Popovich IVD Technology, 2001, 7, 36-42. Oxidation of 7-Deazaguanine: Mismatch-Dependent Electrochemistry and Selective Strand Scission. I. V. Yang, H. H. Thorp Inorg. Chem., 2001, 40, 1690-1697. Oxidation Kinetics of Guanine in DNA Molecules Adsorbed to Indium Tin Oxide Electrodes. P. M. Armistead, H. H. Thorp Anal. Chem., 2001, 73, 558-564. Proton-Coupled Electron Transfer in Duplex DNA: Driving Force Dependence and Isotope Effects on Electrocatalytic Oxidation of Guanine. S. C. Weatherly, I. V. Yang, H. H. Thorp J. Am. Chem. Soc., 2001, 123, 1236-1237. Effects of Base Stacking on Guanine Electron Transfer: Rate Constants for G and GG Sequences of Oligonucleotides from Catalytic Electrochemistry. M. F. Sistare, S. J. Codden, G. Heimlich, H. H. Thorp J. Am. Chem. Soc., 2000, 122, 4742-4749. Electrocatalysis of Guanine Electron Transfer: New Insights from Submillimeter Carbon Electrodes. V. A. Szalai, H. H. Thorp J. Phys. Chem. B., 2000, 104, 6851-6859. Electron Transfer in Tetrads: Adjacent Guanines are not Hole Traps in G Quartets. V. A. Szalai, H. H. Thorp J. Am. Chem. Soc., 2000,122, 4524-4525. Kinetics of Metal-Mediated, One-Electron Oxidation of Guanine in Polymeric DNA and Oligonucleotides Containing Trinucleotide Repeat Sequences. I. V. Yang, H. H. Thorp Inorg. Chem., 2000, 39, 4969-4976. Modification of Metal Oxides with Nucleic Acids: Detection of Attomole Quantities of Immobilized DNA by Electrocatalysis. P. M. Armistead, H. H. Thorp Anal. Chem., 2000, 72, 3764-3770. Electrochemical Detection of Single-Stranded DNA using Polymer-Modified Electrodes. A. C. Ontko, P. M. Armistead, S. R. Kircus, H. H. Thorp Inorg. Chem., 1999, 38, 1842-1846. Electrocatalytic Oxidation of Nucleic Acids at Electrodes Modified with Nylon and Nitrocellulose Membranes. Mary E. Napier and H. Holden Thorp J. Fluorescence, 1999, 9:181-186. Electrochemical Studies of Polynucleotide Binding and Oxidation by Metal Complexes: Effects of Scan Rate, Concentration, and Sequence. M. F. Sistare, R. C. Holmberg, H. H. Thorp J. Phys. Chem. B, 1999, 103, 10718-10728. Site-Selective Electron Transfer from Purines to Electrocatalysts: Voltammetric Detection of a Biologically Relevant Deletion in Hybridized DNA Duplexes. Patricia A. Ropp and H. Holden Thorp Chem. and Biol., 1999. Electrochemical Detection of Single-Stranded DNA using Polymer-Modified Electrodes. A. C. Ontko, P. M. Armistead, S. R. Kircus,-H. H. Thorp Inorg. Chem. 1999, 38, 1842-1846. Electrocatalytic Oxidation of Nucleic Acids at Electrodes Modified with Nylon and Nitrocellulose Membranes. Mary E. Napier and H. Holden Thorp J. Fluorescence 1999, 9:181-186. Electrochemical Studies of Polynucleotide Binding and Oxidation by Metal Complexes: Effects of Scan Rate, Concentration, and Sequence. M. F. Sistare, R. C. Holmberg, H. H. Thorp J. Phys. Chem. B 1999,103, 10718-10728. Site-Selective Electron Transfer from Purines to Electrocatalysts: Voltammetric Detection of a Biologically Relevant Deletion in Hybridized DNA Duplexes. Patricia A. Ropp and H. Holden Thorp Chem. and Biol. 1999, 6:599-605. Cutting Out the Middleman: DNA Biosensors Based on Electrochemical Oxidation. H.H. Thorp Trends in Biotechnol. 1998,16:117-121. Probing Biomolecule Recognition with Electron Transfer: Electrochemical Sensors for DNA Hybridization. M. E. Napier, C. R. Loomis, M. F. Sistare, J. Kim, A. E. Eckhardt and H. H. Thorp Bioconjugate Chem. 1997, 8:996-913. Cyclic Voltammetry Studies of Polynucleotide Binding and Oxidation by Metal Complexes: Homogenouos Electron-Transfer Kinetics. D. H. Johnston, H. H. Thorp J. Phys. Chem. 1996,100,13837-13843. Electrochemical Measurement of the Solvent Accessibility of Nucleobases Using Electron Transfer Between DNA and Metal Complexes. D. H. Johnston, K. C. Glasgow, H. H. Thorp J. Am. Chem. Soc. 1995, 117, 8933 B 8937.) The aforementioned references are hereby incorporated by reference. The following U.S. Patents also describe the Xanthon™ technology and are here by incorporated by reference: U.S. Pat. No. 6,180,346, Electropolymerizable Film, and Method of Making and Use Thereof; U.S. Pat. No. 6,132,971, Electrochemical Detection of Nucleic Acid Hybridization; U.S. Pat. No. 6,127,127, Monolayer and Electrode For Detecting A Label-Bearing Target And Method Of Use Thereof; U.S. Pat. No. 5,968,745, Polymer Electrodes for Detecting Nucleic Acid Hybridization and Method of Use Thereof; U.S. Pat. No. 5,871,918, Electrochemical Detection of Nucleic Acid Hybridization; U.S. Pat. No. 5,171,853, Process of Cleaving Nucleic Acids with Oxoruthenium (IV) Complexes.

As those in the art will appreciate, the size of the array will vary. Arrays containing from about 2 different capture probes to many millions can be made, with very large arrays being possible. Preferred arrays generally range from about 25different capture probes to about 100,000, depending on array composition, with array densities varying accordingly. In a preferred embodiment, the capture probe is attached at both ends. An in another preferred embodiment, capture probes only attached at one end, either 3′ or 5′ end.

Generally, the capture probe allows the attachment of a target analyte to the detection array for the purposes of detection. As is more fully outlined below, attachment of the target analyte to the capture robe may be direct (i.e. the target sequence binds to the capture probe) or indirect (one or more capture extender ligands may be used).

In general, the arrays comprise a substrate with associated capture probes.

The rolling circle primer is an oligonucleotide which anneals to the circularized probe allowing a DNA polymerase to attach to the circularized probe. The rolling circle primer complementary sequence and its cognate primer may have any designed sequence as long as they are complementary to each other but not complementary to other sequences of the probe. Having a primer complementary sequence which is between 15-20 bases long helps ensure that the primer will be sufficiently long to have a unique sequence and hybridize selectively to the probe.

The invention provides precircle probes comprising a number of components, including, but not limited to, targeting domains, cleavage site(s) and labeling sequences. As is known in the art, these precircle probes (and the primers and capture probes outlined herein) can be made in a variety of ways. They may be may be synthesized chemically, e.g., according to the solid phase phosphoramidite triester method described by Beaucage and Caruthers (1981), Tetrahedron Letts., 22(20):1859-1862, e.g., using an automated synthesizer, as described in Needham-VanDevanter et al. (1984) Nucleic Acids Res., 12:6159-6168. Oligonucleotides can also be custom made and ordered from a variety of commercial sources known to persons of skill. Purification of oligonucleotides, where necessary, is typically performed by either native acrylamide gel electrophoresis or by anion-exchange HPLC as described in Pearson and Regnier (1983) J. Chrom. 255:137-149. The sequence of the synthetic oligonucleotides can be verified using the chemical degradation method of Maxam and Gilbert (1980) in Grossman and Moldave (eds.) Academic Press, NY, Methods in Enzymology 65:499-560. Custom oligos can also easily be ordered from a variety of commercial sources known to persons of skill.

In a preferred embodiment, the precircle probes can also comprise additional elements. As is outlined herein, a labeling sequence may also be used. A labeling sequence has substantial complementarity to a label probe comprising labels, that can be added to the amplicons to label them, as is more fully outlined below. Again, it is preferred to use “universal” labeling sequences, or sets of sequences, to minimize the amount of sequence synthesis required and simplify multiplexing using multiple probes and/or multiple targets.

Where probes are prepared by synthetic methods, it may be necessary to phosphorylate the 5′ end of the probe, since oligonucleotide synthesizers do not usually produce oligonucleotides having a phosphate at their 5′ end. The absence of a phosphate at the 5′ end of the probe would otherwise prevent ligation of the 5′ and 3′ ends of the probe. Phosphorylation may be carried out according to methods well known in the art, e.g., using T4 polynucleotide kinase as described, e.g., in U.S. Pat. No. 5,593,840.

Probes and primers can also be prepared by recombinant methods, such as by including the probe in a plasmid that can be replicated in a host cell, e.g., bacteria, amplified and isolated by methods known in the art. The probe can then be cut out of the plasmid using a restriction enzyme that cuts around the probe. Alternatively, large amounts of probe can be prepared by PCR amplification using primers that are complementary to the 5′ and 3′ ends of the probe. The probe can then be further purified according to methods known in the art.

Probes can be prepared in one step, e.g., by synthetically synthesizing the whole probe. Alternatively, probes can be synthesized in at least two parts and linked together through linking oligonucleotides. For example, two parts of a precircle probe can be synthesized and can be linked together by using a bridging oligonucleotide, which contains sequences that are complementary to part A and part B of the probe. This is further described in Example 7. The bridging oligonucleotide is preferably at least from about 20 to about 50 nucleotides long, e.g., between 30 and 40 nucleotides. The bridging oligonucleotide preferably comprises at least about 10, more preferably, at least about 15 or 20 nucleotides that are complementary to each of part A and part B of the probe. The criteria to consider when designing bridging oligonucleotides are the same as those involved in designing a primer for hybridizing to a particular sequence, as described above. The ligation in the presence of the bridging oligonucleotide can be performed by regular ligation methods.

Once the precircle probes have been ligated to form circularized probes, the circles may be continuously transcribed to form tandem-sequence DNA. This is done by adding a rolling circle primer, and extending from the primer using a polymerase. As noted, the rolling circle primer is an oligonucleotide, 15-30 bases long, which will anneal to a complementary region on the circularized probes. The primer is not complementary to any other sequence of the circularized probes and will form a specific and stable duplex with the circularized probes. To aid in the transcription of the circularized probes, the primer may be designed such that the 5′ end has a 4-10 nucleotide sequence which is not complementary to the circularized probes. This non-complementary region of the primer will aid in strand displacement during replication. Including a compatible helicase with the polymerase will also facilitate strand displacement by uncoiling the nucleic acid being amplified. Once the primer has annealed onto the amplification target circle, a DNA polymerase will attach at the site of the replication primer and extend. Tandem-sequence DNA is generated by the DNA polymerase repeatedly copying the circularized probes. The assay mixture may be optimized for the DNA polymerase selected. This reaction mixture should contain deoxynucleoside triphosphates as well as Mg++. The DNA polymerase selected should be a highly processive enzyme. The tandem-sequence DNA which is generated will be a concatamer consisting of repeated transcripts complementary to the circularized probes.

The methods of the invention proceed with the addition of the precircle probes to the target sequence. The targeting domains of the precircle probes hybridize to the target domains of the target sequence. If gaps exist, the reaction proceeds with the addition of one or more NTPs and an extension enzyme (or a gap oligo, as described herein).

In a preferred embodiment, the template nucleic acids and probe(s) are combined in a reaction mixture together with a ligase, ligase buffer and polymerase. The template and probe(s) are then denatured, e.g., by incubation at 95° C. for about 5 to 10 minutes, and then annealed, e.g., by decreasing the temperature of the reaction. As described above, the annealing conditions will depend on the Tm of the homology regions. Polymerization and ligation are then done by adding nucleotides followed by incubation, e.g., for about 10 minutes at 65° C. Alternatively, the nucleic acids are first incubated together in the absence of enzymes, denatured and annealed and then the enzymes are added and the reactions are further incubated for, e.g., about 10 minutes at 65° C.

In order to decrease background signals that result from the attachment and ligation of a non complementary nucleotide, instead of adding a single dNTP to the polymerization reaction, one dNTP could be added along with the other three ddNTP=s. These ddNTPs would not allow ligation but would render the reaction insensitive to small amounts of contaminating nucleotide.

Background signals may also result from-the presence of the “correct” nucleotide in the reaction due to the presence of nucleotides in reagents, and its attachment to the probe. Contamination of reagents with nucleotides can be reduced by treatment of the reagents with an enzyme that degrades free nucleotides. Preferred enzymes include Apyrase and phosphotases, with the former being especially preferred. As described in the Examples, Apyrase is usually added to the reaction prior to the addition of the one or more dNTPs, at about a concentration of 0.5 mU/ul in a typical reaction of about 20 ul. Generally, the reactions are then incubated at 20° C. for a few minutes to up to 30 minutes. The enzyme is then denatured by incubation of the reaction for about 5 to 10 minutes at 95° C. Alternatively alkaline phosphatases may be used such as, e.g. shrimp alkaline phosphatase.

Ligation of the 3′ and 5′ ends of the probe(s) can be performed using an enzyme, or chemically. Preferably, ligation is carried out enzymatically using a ligase in a standard protocol. Many ligases are known and are suitable for use in the invention, e.g. Lehman, Science, 186: 790-797 (1974); Engler et al, DNA Ligases, pages 3-30 in Boyer, editor, The Enzymes, Vol. 15B (Academic Press, New York, 1982); and the like. Preferred ligases include T4 DNA ligase, T7 DNA ligase, E. coli DNA ligase, Taq ligase, Pfu ligase, and Tth ligase. Protocols for their use are well known, e.g. Sambrook et al (cited above); Barany, PCR

Methods an Applications, 1: 5-16 (1991); Marsh et al, Strategies, 5: 73-76 (1992); and the like. Generally, ligases require that a 5′ phosphate group be present for ligation to the 3′ hydroxyl of an abutting strand. Preferred ligases include thermostable or (thermophilic) ligases, such as pfu ligase, Tth ligase, Taq ligase and Ampligase TM DNA ligase (Epicentre Technologies, Madison, Wis.). Ampligase has a low blunt end ligation activity.

The preferred ligase is one which has the least mismatch ligation and ligation across the gap activity. The specificity of ligase can be increased by substituting the more specific NAD+-dependant ligases such as E. coli ligase and (thermostable) Taq ligase for the less specific T4 DNA ligase. The use of NAD analogues in the ligation reaction further increases specificity of the ligation reaction. See, U.S. Pat. No. 5,508,179 to Wallace et al.

The conditions for carrying out the ligation will depend on the particular ligase used and will generally follow the manufacturer=s recommendations. For example, preferred Ampligase concentrations are from about 0.0001 to about 0.001 u/ul, and preferably about 0.0005 u/ul. Preferred concentrations of probe nucleic acids are from about 0.001 to about 0.01 picomoles/ul and even more preferably, about 0.015 picomoles/ul. Preferred concentrations of template nucleic acids include from about 1 zeptomole/ul to about 1 attomole/ul, most preferably about 5 zeptomoles/ul. A typical reaction is performed in a total of about 20 ul.

In a preferred embodiment, the template nucleic acids and probe(s) are combined in a reaction mixture together with a ligase and ligase buffer. The template and probe(s) are then denatured, e.g., by incubation at 95° C. for about 5 to 10 minutes, and then annealed, e.g., by decreasing the temperature of the reaction. The annealing conditions will depend on the Tm of the homology regions, as described elsewhere herein. Annealing can be carried out by slowing reducing the temperature from 95° C. to about the Tm or several degrees below the Tm. Alternatively, annealing can be carried out by incubating the reaction at a temperature several degrees below the Tm for, e.g., about 10 to about 60 minutes. For example, the annealing step can be carried out for about 15 minutes. Ligation can be then carried out by incubation the reactions for about 10 minutes at 65° C.

Alternatively, the nucleic acids are denatured and annealed in the absence of the ligase, and the ligase is added to the annealed nucleic acids and then incubated, e.g., for about 10 minutes at 65° C. This embodiment is preferably for non heat stable ligases.

As mentioned previously, unreacted probes can contribute to backgrounds from undesired non-specific amplification. In a preferred embodiment, any unreacted precircle probes and/or target sequences are rendered unavailable for amplification. This can be done in a variety of ways, as will be appreciated by those in the art. In one embodiment, exonucleases are added, that will degrade any linear nucleic acids, leaving the closed circular probes. Suitable 3′-exonucleases include, but are not limited to, exo I, exo III, exo VII, exo V, and polymerases, as many polymerases have excellent exonuclease activity, etc.

By “extension enzyme” herein is meant an enzyme that will extend a sequence by the addition of NTPs. As is well known in the art, there are a wide variety of suitable extension enzymes, of which polymerases (both RNA and DNA, depending on the composition of the target sequence and precircle probe) are preferred. Preferred polymerases are those that lack strand displacement activity, such that they will be capable of adding only the necessary bases at the end of the probe, without further extending the probe to include nucleotides that are complementary to a targeting domain and thus preventing circularization. Suitable polymerases include, but are not limited to, both DNA and RNA polymerases, including the Klenow fragment of DNA polymerase I, SEQUENASE 1.0 and SEQUENASE 2.0 (U.S. Biochemical), T5 DNA polymerase, Phi29 DNA polymerase and various RNA polymerases such as from Thermus sp., or Q beta replicase from bacteriophage, also SP6, T3, T4 and T7 RNA polymerases can be used, among others.

Even more preferred polymerases are those that are essentially devoid of a 5′ to 3′ exonuclease activity, so as to assure that the probe will not be extended past the 5=end of the probe. Exemplary enzymes lacking 5=to 3=exonuclease activity include the Klenow fragment of the DNA Polymerase and the Stoffel fragment of DNAPTaq Polymerase. For example, the Stoffel fragment of Taq DNA polymerase lacks 5′ to 3′ exonuclease activity due to genetic manipulations, which result in the production of a truncated protein lacking the N-terminal 289 amino acids. (See e.g., Lawyer et al., J. Biol. Chem., 264:6427-6437 [1989]; and Lawyer et al., PCR Meth. Appl., 2:275-287 [1993]). Analogous mutant polymerases have been generated for polymerases derived from T. maritima, Tsps17, TZ05, Tth and Taf.

Preferred polymerases are those that lack a 3′ to 5′ exonuclease activity, which is commonly referred to as a proof-reading activity, and which removes bases which are mismatched at the 3′ end of a primer-template duplex. Although the presence of 3′ to 5′ exonuclease activity provides increased fidelity in the starnd synthesized, the 3′ to 5′ exonuclease activity found in thermostable DNA polymerases such as Tma (including mutant forms of Tma that lack 5′ to 3′ exonuclease activity) also degrades single-stranded DNA such as the primers used in the PCR, single-stranded templates and single-stranded PCR products. The integrity of the 3′ end of an oligonucleotide primer used in a primer extension process is critical as it is from this terminus that extension of the nascent strand begins. Degradation of the 3′ end leads to a shortened oligonucleotide which in turn results in a loss of specificity in the priming reaction (i.e., the shorter the primer the more likely it becomes that spurious or non-specific priming will occur).

Preferred polymerases are thermostable polymerases. For the purposes of this invention, a heat resistant enzyme is defined as any enzyme that retains most of its activity after one hour at 40° C. under optimal conditions. Examples of thermostable polymerase which lack both 5′ to 3′ exonuclease and 3′ to 5′ exonuclease include Stoffel fragment of Taq DNA polymerase. This polymerase lacks the 5′ to 3′ exonuclease activity due to genetic manipulation and no 3′ to 5′ activity is present as Taq polymerase is naturally lacking in 3′ to 5′ exonuclease activity. Tth DNA polymerase is derived form Thermus thermophilus, and is available form Epicentre Technologies, Molecular Biology Resource Inc., or Perkin-Elmer Corp. Other useful DNA polymerases which lack 3′ exonuclease activity include a Vent[R](exo-), available from New England Biolabs, Inc., (purified from strains of E. coli that carry a DNA polymerase gene from the archaebacterium Thermococcus litoralis), and Hot Tub DNA polymerase derived from Thermus flavus and available from Amersham Corporation.

Other preferred enzymes which are thermostable and deprived of 5′ to 3′ exonuclease activity and of 3′ to 5′ exonuclease activity include AmpliTaq Gold. Other DNA polymerases, which are at least substantially equivalent may be used like other N-terminally truncated Thermus aquaticus (Taq) DNA polymerase I. the polymerase named KlenTaq I and KlenTaq LA are quite suitable for that purpose. Of course, any other polymerase having these characteristics can also be used according to the invention.

The conditions for performing the addition of one or more nucleotides at the 3′ end of the probe will depend on the particular enzyme used, and will generally follow the conditions recommended by the manufacturer of the enzymes used.

The nucleotides are preferably added to a final concentration from about 0.01 uM to about 100 uM, and preferably about 0.1 UM to 10 UM in the reaction. The concentration of ligase to add is described in the following section. Preferred amounts of Taq DNA Polymerase Stoffel fragment include 0.05 u/ul. A typical reaction volume is about 10 to 20 ul. Preferred amounts of template and probe DNA are also described in the following section.

One of skill in the art will recognize that subsequent analysis and detection of the amplification products may be done in a variety of ways. Detection labels such as radioactive isotopes, fluorescent molecules, phosphorescent molecules, enzymes, antibodies, ligands, etc. may also be incorporated directly into the amplification products, or alternatively can be coupled to detection molecules for subsequent detection and analysis. Preferred methods include chemiluminescence, using both Horseradish Peroxidase and/or Alkaline Phosphatase with substrates that produce photons as breakdown products (kits available from Amersham, Boehringer-Mannheim, and Life Technologies/Gibco BRL); color production using both Horseradish Peroxidase and/or Alkaline Phosphatase with substrates that produce a colored precipitate (kits available from Life Technologies/Gibco BRL, and Boehringer-Mannheim); chemifluorescence using Alkaline Phosphatase and the substrate AttoPhosJ Amersham or other substrates that produce fluorescent products; fluorescence using Cy-5 (Amersham), fluorescein, and other fluorescent tags; radioactivity using end-labeling, nick translation, random priming, or PCR to incorporate radioactive molecules into the ligation oligonucleotide or amplification product. Other methods for labeling and detection will be readily apparent to one skilled in the art.

In one embodiment, the detection labels are incorporated directly into the amplification products during rolling circle amplification of the closed circular target. Examples of detection labels that can be incorporated into amplified DNA or RNA include nucleotide analogs such as BrdUrd (Hoy and Schimke, Mutation Research 290:217-230 (1993)), BrUTP (Wasnick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res. 22:3226-3232 (1994)). A preferred nucleotide analog detection label for DNA is BrdUrd (BUDR triphosphate, Sigma), and a preferred nucleotide analog detection label for RNA is Biotin-16-uridine-5′-triphosphate (Biotin-16-dUTP, Boehringher Mannheim). Molecules that combine two or more of these detection labels are also contemplated for use in the disclosed methods.

Detection labels that are incorporated into amplified nucleic acid, such as biotin, can be subsequently detected using sensitive methods well-known in the art. For example, biotin can be detected using streptavidin-alkaline phosphatase conjugate (Tropix, Ind.), which is bound to the biotin and subsequently detected by chemiluminescence of suitable substrates (for example, chemiluminescence substrate CSPD; disodium, 3-(4-methoxyspiro-[1,2-dioxetane-3-2′(5′-chloro)tricyclo [3.3.1.1^{3.7}] decane]-4-yl) phenyl phosphate; Tropix, Inc.). A preferred detection label for use in detection of amplified RNA is acridinium-ester-labeled DNA probe (GenProbe, Inc., as described by Arnold et al., Clinical Chemistry 35:1588-1594 (1989)). An acridinium-ester-labeled detection probe permits the detection of amplified RNA without washing because unhybridized probe can be destroyed with alkali (Arnold et al. (1989)).

Another embodiment utilizes a detection probe labeled with any composition detectable by spectroscopic, photochemical, biochemical, immunochemical, electrical, optical or chemical means. Preferred labels in the present invention include spectral labels such as fluorescent dyes (e.g., fluorescein isothiocyanate, Texas red, rhodamine, dixogenin, biotin, and the like), radiolabels (e.g., ³H, ¹²⁵I, ³⁵S, ¹⁴C, ³²P, ³³P, etc.), enzymes (e.g., horse-radish peroxidase, alkaline phosphatase, etc.), spectral calorimetric labels such as colloidal gold or colored glass or plastic (e.g. polystyrene, polypropylene, latex, etc.) beads. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. Thus, a wide variety of labels may be used, with the choice of label depending on sensitivity required, ease of conjugation with the compound, stability requirements, available instrumentation, and disposal provisions.

The label may be coupled directly or indirectly to the molecule to be detected according to methods well known in the art. Non-radioactive labels are often attached by indirect means. Generally, a ligand molecule (e.g., biotin) is covalently bound to a nucleic acid such as a probe, primer, amplicon, YAC, BAC or the like. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with labeled, anti-ligands. Alternatively, any haptenic or antigenic compound can be used in combination with an antibody. Labels can also be conjugated directly to signal generating compounds, e.g., by conjugation with an enzyme or fluorophore or chromophore.

Means of detecting labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes and many other detection systems which are widely available. In general, a detector which monitors a probe-target nucleic acid hybridization is adapted to the particular label which is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising a nucleic acid array with particular set of probes bound to the array is digitized for subsequent computer analysis.

Fluorescent labels are preferred labels, having the advantage of requiring fewer precautions in handling, and being amendable to high-throughput visualization techniques. Preferred labels are typically characterized by one or more of the following: high sensitivity, high stability, low background, low environmental sensitivity and high specificity in labeling. Fluorescent moieties, which are incorporated into the labels of the invention, are generally are known, including Texas red, dixogenin, biotin, 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidazolylphenylamine, 2-oxo-3-chromen, indole, xanthen, 7-hydroxycoumarin, phenoxazine, calicylate, strophanthidin, porphyrins, triarylmethanes and flavin. Individual fluorescent compounds which have functionalities for linking to an element desirably detected in an apparatus or assay of the invention, or which can be modified to incorporate such functionalities include, e.g., dansyl chloride; fluoresceins such as 3,6-dihydroxy-9-phenylxanthydrol; rhodamineisothiocyanate; N-phenyl 1-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl-N-methyl-2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine: N,N′-dihexyl oxacarbocyanine; merocyanine, 4-(3′-pyrenyl)stearate; d-3-aminodesoxy-equilenin; 12-(9′-anthroyl)stearate; 2-methylanthracene; 9-vinylanthracene; 2,2′(vinylene-p-phenylene)bisbenzoxazole; p-bis(2- -methyl-5-phenyl-oxazolyl))benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N-(p-(2benzimidazolyl)-phenyl)maleimide; N-(4-fluoranthyl)maleimide; bis(homovanillic acid); resazarin; 4-chloro-7-nitro-2,1,3-benzooxadiazole; merocyanine 540; resorufin; rose bengal; and 2,4-diphenyl-3(2H)-furanone. Many fluorescent tags are commercially available from SIGMA chemical company (Saint Louis, Mo.), Molecular Probes, R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.) as well as other commercial sources known to one of skill.

In a preferred embodiment, the amplification products obtained following the methods of the present invention are detected using conventional sequence-specific probe technology, such as the cross-linkable capture and reported probes described in U.S. Pat. Nos. 6,277,570; 6,005,093 and 6,187,532, the disclosures of which are incorporated by reference herein.

In another preferred embodiment, molecular beacons are employed as described in Leone et al., Nuc. Acids Res. 26:2150-55 (1995); Tyagi et al., Nature Biotech. 14:303-308 (1996); Kostritis et al., Science 279:1228-29 (1998); Tyagi et al. Nature Biotech. 16:49-53 (1998); Vet et al. Proc. Nat. Acad. Sci. USA 96:6394-99 (1999) and Marras et al., Genet. Anal. Biomol. Eng. 14:151-156 (1999). Briefly, molecular beacons are dual-labeled oligonucleotides having a fluorescent reported group at one end and a fluorescent quencher group at the other end, which in the absence of target form an internal hairpin that brings the reported and quencher in physical proximity so as to quench the flourescent signal. In the presence of target, the probe molecule unfolds and hybridizes to the target, resulting in separation of the reporter and quencher and emission of a fluorescent signal upon stimulation. In preferred embodiments, the quencher comprises Dabcyl (4-(4′-dimethylaminophenylazo)benzoic acid) and the fluorophore comprises fluorescein, tetrachloro-6-carboxyfluorescein, hetra-6-carboxyfluorescein, tetramethylrhodamine or rhodamine-X. Alternatively, detection techniques such as fluorescence resonance energy transfer (FRET) (Ota et al., Nuc. Acids. Res. 26:735-43 (1998)) and TaqManJ (Livak et al., PCR Methods Appl. 4:357-62 (1995); Livak, Genet. Anal. 14:143-49 (1999); Chen et al., J. Med. Virol. 65:250-56(2001)) can be employed

In an alternative embodiment, the circular targets are detected on a micro-formatted multiplex or matrix devices (e.g., DNA chips) (see M. Barinaga, 253 Science, pp. 1489, 1991; W. Bains, 10 Bio/Technology, pp. 757-758, 1992). These methods usually attach specific DNA sequences to very small specific areas of a solid support, such as micro-wells of a DNA chip. In one variant, the invention is adapted to solid phase arrays for the rapid and specific detection of multiple polymorphic nucleotides, e.g., SNPs. Typically, an oligonucleotide such as the ligation oligonucleotide of the present invention is linked to a solid support and a target nucleic acid is hybridized to the oligonucleotide. Either the oligonucleotide, or the target, or both, can be labeled, typically with a fluorophore. Where the target is labeled, hybridization is detected by detecting bound fluorescence. Where the oligonucleotide is labeled, hybridization is typically detected by quenching of the label. Where both the oligonucleotide and the target are labeled, detection of hybridization is typically performed by monitoring a color shift resulting from proximity of the two bound labels. A variety of labeling strategies, labels, and the like, particularly for fluorescent based applications are described, supra.

In one embodiment, an array of ligation oligonucleotides are synthesized on a solid support. Exemplar solid supports include glass, plastics, polymers, metals, metalloids, ceramics, organics, etc. Using chip masking technologies and photoprotective chemistry it is possible to generate ordered arrays of nucleic acid probes. These arrays, which are known, e.g., as “DNA chips.”

The construction and use of solid phase nucleic acid arrays to detect target nucleic acids is well described in the literature. See, Fodor et al. (1991) Science, 251: 767-777; Sheldon et al. (1993) Clinical Chemistry 39(4): 718-719; Kozal et al. (1996) Nature Medicine 2(7): 753-759 and Hubbell U.S. Pat. No. 5,571,639. See also, Pinkel et al. PCT/US95/16155 (WO 96/17958). In brief, a combinatorial strategy allows for the synthesis of arrays containing a large number of probes using a minimal number of synthetic steps. For instance, it is possible to synthesize and attach all possible DNA 8 mer oligonucleotides (65,536 possible combinations) using only 32 chemical synthetic steps. In general, VLSIPS TM procedures provide a method of producing 4ⁿ different oligonucleotide probes on an array using only 4n synthetic steps.

Light-directed combinatorial synthesis of oligonucleotide arrays on a glass surface is performed with automated phosphoramidite chemistry and chip masking techniques similar to photoresist technologies in the computer chip industry. Typically, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface.

Thus, the compositions of the present invention may be used in a variety of research, clinical, quality control, or field testing settings.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLES

### Example 1

### SNP Genotyping with RCA Signal Amplification on Hydrogel Microarrays

Strategies for universal, on-chip rolling circle amplification (RCA) of genotyping signals generated by single base extension (SBE) of immobilized oligonucleotides on hydrogel-based microarrays are described. RCA technology was successful in achieving a 3-log enhancement of SBE signals with SNP target detection limits of 4 pM. Allele discrimination ratios of 5 to 30 were achieved with homozygous targets over a 2-log range of target concentrations, with signal-to-noise ratios ranging from 5 to 25 for a set of six SNP-containing duplex DNA amplicon targets. The sensitivity of SBE-RCA with unmodified human genomic DNA target using SBE probes designed from repetitive sequence element families varying in abundance from 10³ copies or greater per haploid genome was similar to that with PCR amplicons with single copy sequences (4 pM). These studies demonstrate the compatibility of universal RCA signal amplification with hydrogel microarrays as well as with SBE allele discrimination, and emphasize the utility of RCAT signal amplification in developing high throughput, cost effective, population scale SNP genotyping applications.

The completion of a reference sequence database of the human genome has catalyzed renewed interest in diverse fields of study involving gene-based medicine, including pharmacogenomics, diagnostics, and therapeutics. The next major effort in the field of genetic analysis in humans is to obtain a more detailed understanding of the clinical significance of nucleic acid sequence variation among populations. Single nucleotide polymorphisms (SNPs) are predominantly bi-allelelic variants that occur in the human genome about once every kilobase (Kruglyak, L. (1999) Nat Genet 22: 139-44), and are therefore the markers of choice for a wide variety of genetic studies.

Alutomated DNA sequencing techniques were effectively employed for the determination of the reference human genome sequence. However, the practical task of profiling polymorphic variants in human populations must take other technological formats. Microarrays of immobilized oligonucleotide reagents provide a rapid, inexpensive, miniaturized, scalable and automatable platform for large-scale SNP genotyping applications. A variety of microarray substrates and assay strategies for allele discrimination have been described (Southern, EM (2001), Methods Mol Biol 170: 1-15 and Hacia et al. (1999) Nat Genet 22: 164-7). Planar glass arrays with in-situ probe synthesis (Lipshutz et al, (1999) Nat Genet 21 (1 Suppl): 2-04) or off-line, oligonucleotide deposition (Pastinen et al. 1997) has been widely employed.

Single nucleotide differences can be scored using one of two distinct target recognition modes—allele-specific hybridization (Wang et al., (1998) Science 280: 1077-82) or enzymatic recognition (Chen and Kwok (1999) Genet Anal 14: 157-63); Pastinen, et al. (2000) Genome Res 10: 1031-42). Traditionally, genotyping reactions on microarrays and in solution, have involved the use of the polymerase chain reaction (PCR) for target (genetic locus) pre-amplification. PCR has limited multiplexing capability and adds cost and complexity to the assays (Wang et al., (1998) Science 280: 1077-82, Pastinen, et al. (2000) Genome Res 10: 1031-42). Therefore, it is of limited applicability in genome-wide scans or clinical applications employing a large cohort of SNP's.

Universal, on-chip RCA signal amplification provides a high degree of multiplexing and thereby affords template economy for genotyping applications. Due to its unique property of localized product detection with linear kinetics, RCA provides adequate sensitivity needed for direct detection and quantitation of unmodified nucleic acid targets (Nallur et al, (2001) Nucleic Acids Res., 29: E118). Previously, we have demonstrated picomolar sensitivity with oligonucleotide targets by combining on-chip allele-specific ligation with RCA signal amplification on planar as well as porous acrylamide gel arrays (Nallur et al, (2001) Nucleic Acids Res., 29: E118). This report describes the application of RCA for universal amplification of single base extension (SBE) signals on hydrogel microarrays. RCA provided greater than 1000-fold enhancement of genotype-specific signals in multiplexed genotyping assays involving a set of six SNP targets. Uniform signal amplification by RCA resulted in accurate genotyping of each of the SNPs over a 2-log range of target concentrations without measurable bias in the fidelity of SBE allele discrimination. RCA-mediated signal enhancement was similar with PCR products (specific amplicons) and unmodified human genomic DNA.

Results

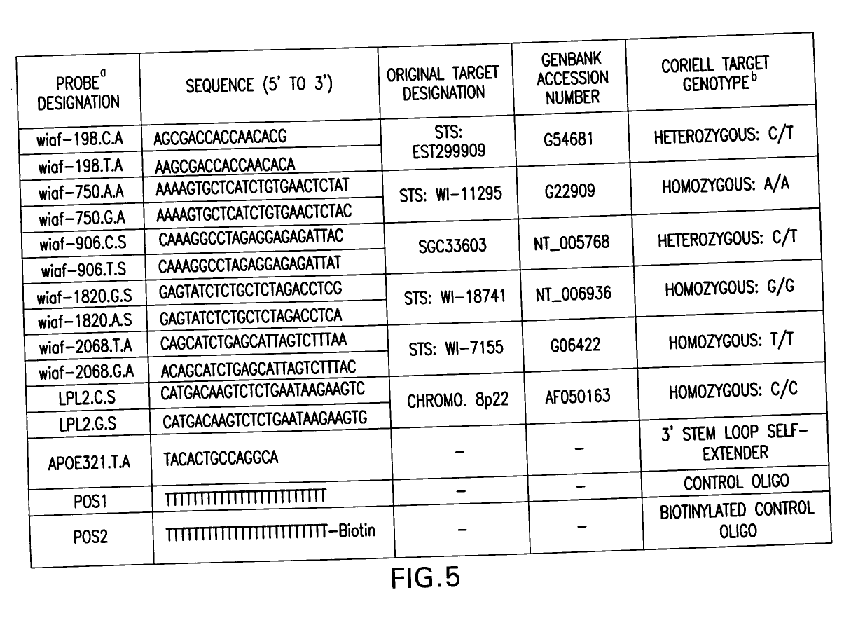

Assays demonstrating amplification of Single Base Extension (SBE) signals using Rolling Circle Amplification (RCA) on hydrogel arrays. The SNP assay employed incorporation of biotin tagged acyclo-nucleoside triphosphate analogs (chain terminating) at the 3′ termini of allele-discriminating oligonucleotide probes by a DNA polymerase (Thermosequenase™, Vendor). Single base extension (SBE) probes contained a common gene specific region for hybridization with the target, but differed by a single, allele-specific, 3′ terminal nucleotide designed to query the identity of the SNP nucleotide in the target. Thus, a pair of allele-specific oligonucleotides was immobilized in adjacent microarray spots for each SNP target. The SBE probe designations and sequences used in this study are presented in FIG. 5.

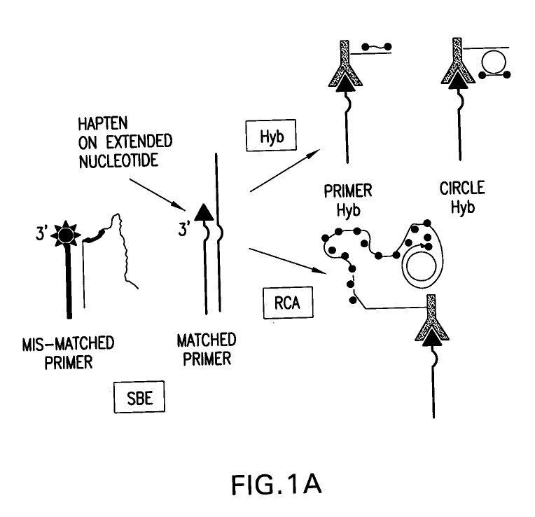

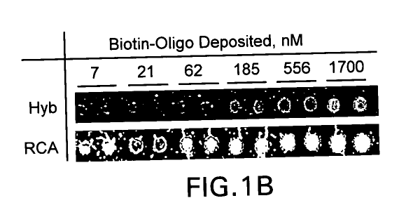

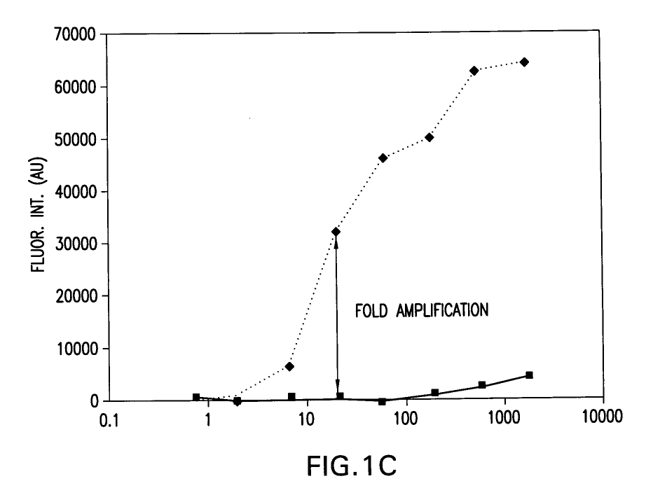

The SBE genotyping chip contained the oligonucleotide probes anchored onto hydrogel substrate at their 5′ ends. (FIG. 1A). In SBE, complementary base pairing of the target with the probe oligonucleotide at it's 3′ terminus supports DNA polymerase-mediated extension resulting in the incorporation of a single biotinylated-nucleotide at the 3′ terminus. The chip-based SBE signals are then amplified and detected by immuno-Rolling Circle Amplification (RCA). In RCA, an a-biotin antibody conjugated to an RCA primer binds to the biotin on the extended SBE probe and serves to anchor the platform for RCA signal amplification (Nallur et al, (2001) Nucleic Acids Res., 29: E118). An RCA amplification circle (Circle 1) is annealed to the conjugated primer and the resultant primer:circle duplex is amplified by RCA. The concatenated RCA product is detected by hybridizing, fluorophore-labeled oligonucleotides (“decorators”) complementary to the RCA product (Nallur et at, (2001) Nucleic Acids :Res., 29: E118). In parallel assays, a decorator probe was hybridized either directly to the antibody-primer conjugate (“primer decorator”), or to the RCA circle (“circle decorator”), pre-annealed to the antibody conjugate, both in the absence of RCA signal amplification (Hyb). RCA and Hyb signals are determined by laser scanner digital fluorometry and quantitated (See Experimental protocol). RCA-mediated signal amplification is determined by taking the ratio of fluorescence intensities of RCA/Hyb.

Detection of biotin-labeled oligonucleotides by RCA on hydrogel microarrays. The compatibility of hydrogel microarrays substrates with RCA signal amplification, as well as the sensitivity with which hydrogel-immobilized biotin-labeled oligonucleotides could be detected by RCA signal amplification were investigated using chips pre-dispensed with oligonucleotides containing biotin moieties. The microarrayed spots in these chips comprised 3′-biotin-labeled oligonucleotides serially diluted with unlabeled oligonucleotides prior to immobilization. The concentration of the biotin-labeled oligonucleotides in the mixture varied over a 2.5×10³-fold range (770 pM to 1.7 μM), while the final oligonucleotide concentration was fixed at 18 μM. RCA was performed with the pre-dispensed chip using an α-biotin antibody-primer1 conjugate, and detected with Cy5-labeled oligonucleotide primer-specific decorators as described (Nallur et al, (2001) Nucleic Acids Res., 29: E118. See also Experimental protocol). The observed limit of detection of the immobilized biotinylated oligonucleotides was 770 pM at a minimal signal-to-noise ratio of 2 (FIGS. 1B and 1C). Under the stated experimental conditions, the level of sensitivity represents detection of 2.3×10⁵ biotinylated oligonucleotides per 200 μm spot, assuming 100% immobilization efficiency. In contrast, the limit of detection of biotinylated oligonucleotides using direct hybridization of the decorators oligonucleotides to the bound conjugate (Hyb) in the absence of RCA was 185 nM. These results represent 240-fold increase in sensitivity of detection of the surface-bound haptens by RCA compared to direct hybridization (FIG. 1C). Nearly 3000-fold RCA signal amplification occurred in the middle of the scanner's linear detection range (achieved at a biotinylated primer concentration of 21 nM). The results also showed a 3 log dynamic range for detection of biotinylated oligonucleotides by RCA signal amplification on hydrogel substrates. These findings suggest that hydrogel substrates support robust RCA signal amplification and provide efficiencies comparable to those obtained with planar arrays (Nallur et al, (2001) Nucleic Acids Res., 29: E118).

From the above results it is clear that at least 2.3×10⁵ oligonucleotide probe molecules would need to be extended in an SBE assay, in order to be detected by SBE-RCA at the limit of sensitivity. Under robust SBE conditions, this number represents a SNP target concentration of 4.8 fM, or 0:5-1.0 μg human genomic DNA to be used per SBE assay. These calculations support the expectation that a coupled SBE-RCA assay should provide sufficient sensitivity for detection of single nucleotide polymorphisms by SBE on hydrogel microarrays using unmodified genomic nucleic acids.

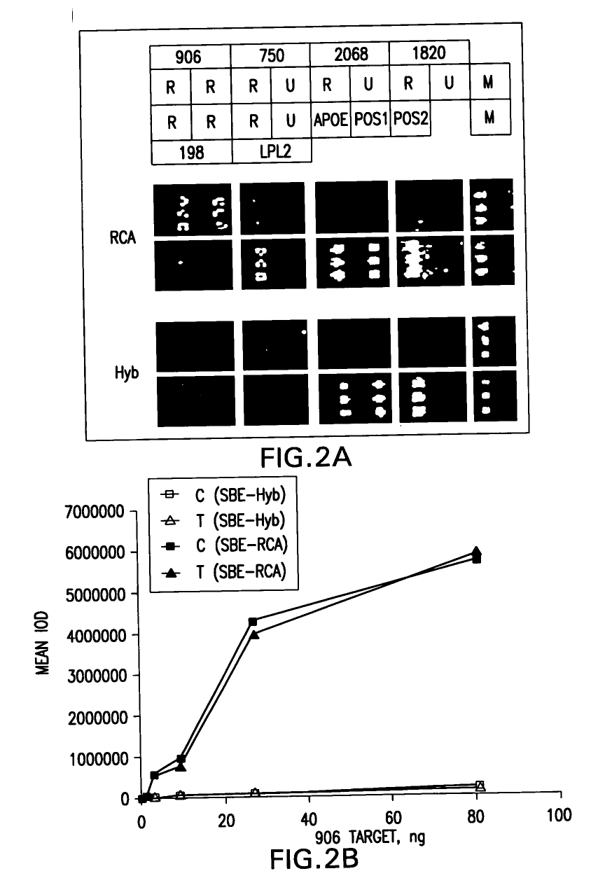

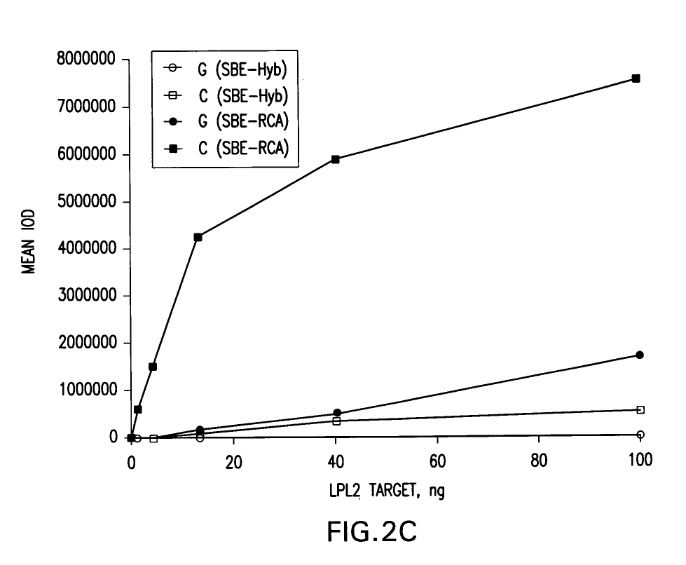

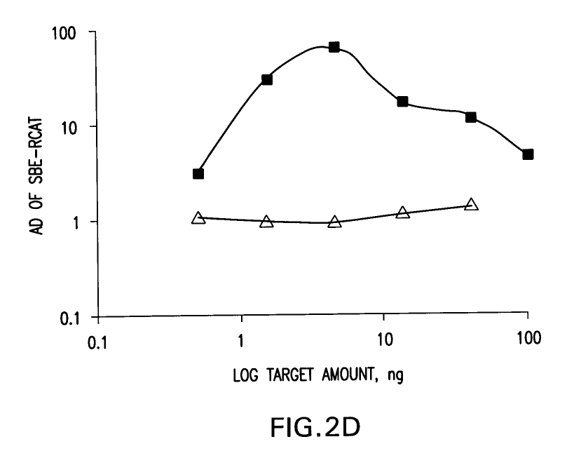

SNP genotyping with RCA signal amplification. The combined SBE-RCA approach was used in genotyping assays on hydrogel microarrays containing immobilized probe pairs for a set of 6 SNP-containing genetic loci were selected from the Whitehead SNP Database (maintained by the Center for Genome Research at the Whitehead Institute for Biomedical Research, Cambridge, Mass., USA). The sequences of the SBE probes used in this study are shown in FIG. 5. Initial SBE assays employed PCR amplified targets derived from genomic DNA obtained from the Coriell Cell Repositories (#:M08PDR, PD0007. See Experimental protocol for detailed information). Genotypes of the DNA samples were confirmed by conventional sequencing techniques (ABI 310, Perkin-Elmer Corporation). Microarray genotyping reactions involved multiplexing of sets of 2-3 SNP amplicon target preparations per SBE assay. The SBE signals were amplified by RCA and the resultant fluorescence intensities were detected as previously described. FIGS. 2A-C depicts the SBE-RCA signals specific for the targets 906 and LPL2, and reflects their respective genotypes (See also FIG. 5). The limit of detection of SNP's in the PCR targets at a signal-to-noise ratio of 2 was 1 ng, which corresponded to 4 pM. For the homozygous target, LPL2, a specific RCA signal was observed for the represented allele (G) whose signal intensity was 20- to 50-fold greater than that for the un-represented allele (FIGS. 2B and 2C). Equivalent SBE-RCA signals were present for both alleles of the heterozygous SNP target, 906, with allele discrimination ratios close to unity over a 2-log range of target concentrations (FIG. 2D) indicating uniform RCA amplification, as well as a lack of sequence-dependent bias for specific amplified signal yield. Hyb signal intensities and target sensitivities for the same targets, were 100- to 150-fold lower than the corresponding signal intensities with RCA signal amplification.

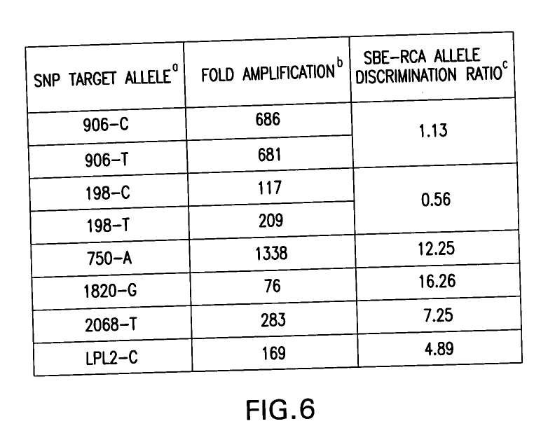

Overall, the panel of 6 SNP targets demonstrated nearly 2-3 logs of SBE signal increase with RCA amplification regardless of the manner in which the SNP targets were multiplexed (FIG. 6). Targets showing low SBE-RCA signal intensities also demonstrated weak SBE signals-(data not shown); suggesting that resultant lower RCA amplification could be due to lower initial SBE product yield. Nevertheless, the approximately 100- to 1000-fold RCA amplification of SBE signals observed for the panel of SNP targets is in agreement with the values obtained from the immobilized, biotinylated oligonucleotide detection studies. This suggests that RCA amplification of SBE signals proceeds optimally on microarrays. In general, the SNP target SBE-RCA assays displayed robust allele discrimination ratios, making it possible to score the genotypes with a high degree of confidence (94.5 to 96.7%). Interestingly, RCA-mediated allele discrimination for the homozygous targets was enhanced 2-5 fold over that obtained with Hyb or SBE alone (data not shown). These observations suggest that RCA signal amplification not only improves the sensitivity of genotyping on microarrays but also may enhance the fidelity of allele discriminating signals. The allele discrimination factors for the heterozygous targets, 906 and 198, remained close to unity, suggesting uniform amplification of SBE signals from both alleles (FIG. 6).

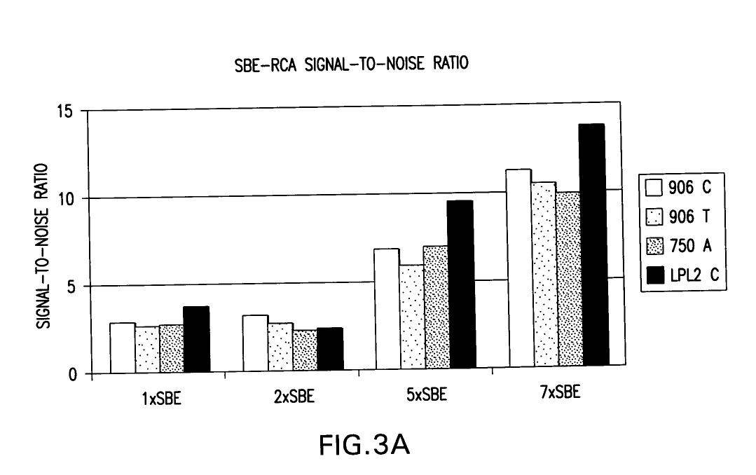

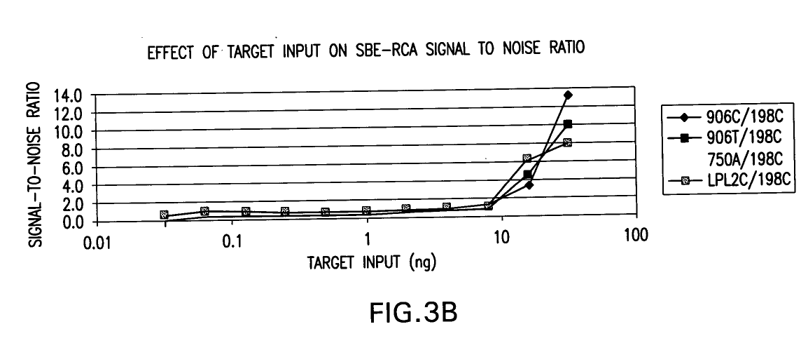

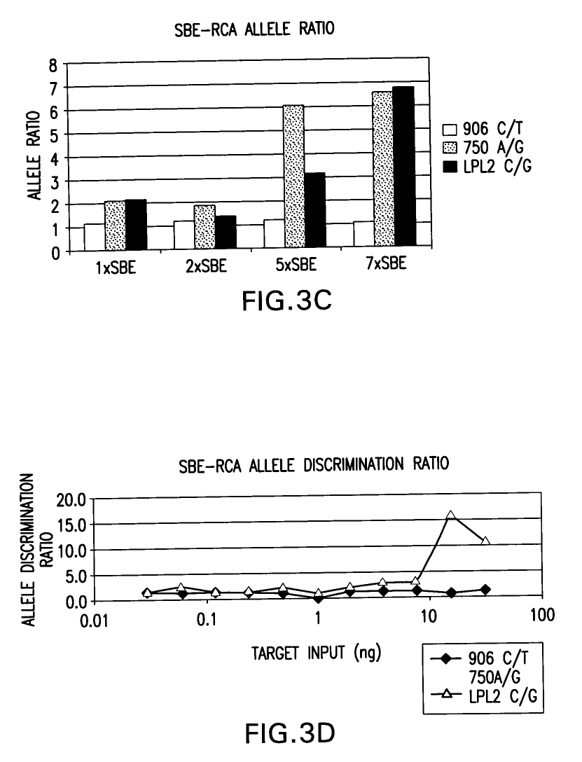

Some assay parameters also affected the practical application of the SBE-RCA genotyping method. Increasing the number of SBE cycles resulted in increased signal-to-noise and allele discrimination ratios for both hetero- and homozygous targets (FIG. 3A). A modest increase in RCA background was observed with increased SBE cycles: The level of target input also affected the allele discrimination and signal-to-noise ratios (FIGS. 3B and 3C). Robust homozygous allele discrimination ratios were achieved above 1-2 ng of amplicon target per 80 μl SBE assay (100-200 pM of ˜100 bp amplicon target). As expected, the allele discrimination ratios of the heterozygous targets were unaffected (FIG. 3C). The signal-to-noise ratios for all targets increased with target input (FIG. 3D); appreciable ratios were achieved with at least 1-3 ng targets per 80 μl reaction. Allele discrimination and signal:noise diminished with decreasing target concentrations with all the targets tested and reached background levels at concentrations below 3-4 pM, at which concentration, greater than 10⁸ target molecules are present in the SBE mix. Perhaps a low SBE yields on account of decreasing hybridization capture or decreased polymerase affinity for available duplexes could account for some of these observations. However, as a whole, these data demonstrate that RCA amplification of SBE signals, on hydrogel microarrays, faithfully replicates signals generated by SBE and that such amplification is substantially free of sequence-dependent biases, both among SNP-containing loci, and SNP alleles.

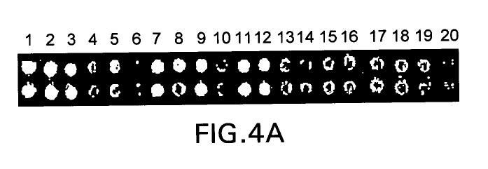

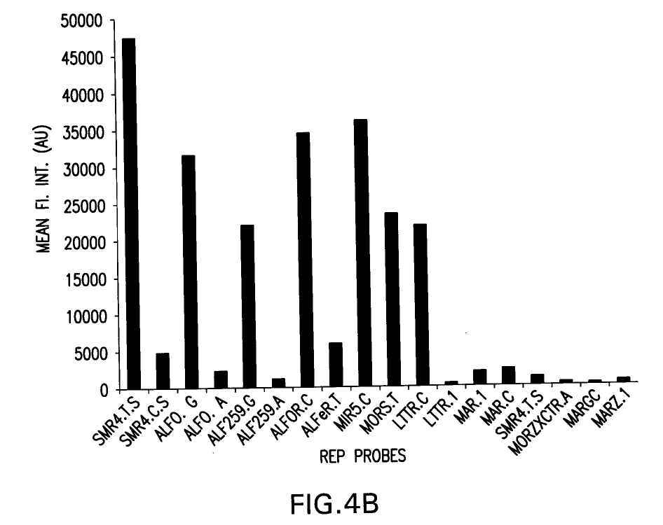

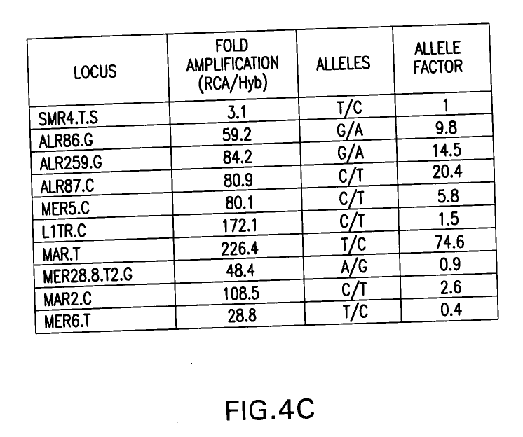

SBE-RCA with unmodified targets. From cost and throughput perspectives, genotyping unmodified genomic DNA templates is an attractive, yet formidable proposition. It is clear from the SBE-RCA sensitivity using PCR amplicons that the ability to perform genotyping with unmodified genomic targets requires at least 2 logs of additional. SBE reactions were performed using fragmented human genomic DNA targets and the signals were amplified by RCA. To measure assay sensitivity, a genotyping chip was designed (GENO1) that contained allele-discriminating SBE probes complementary to each of several families of human repetitive elements and gene families (FIGS. 4A and 4B). The repetitive elements varied in abundance from over a million copies to less than a thousand copies per haploid genome. Following SBE-RCA, probes corresponding to sequences represented at 1000 copies or greater per genome were readily detected (FIG. 4A). The experimental conditions represented a sensitivity of detection of 3 pM with respect to a single copy gene, which corresponded well with the sensitivity observed with PCR amplicons. As with PCR amplicons, RCA signal intensity and the allele discrimination factors decreased with decreasing representation probe-complementary sequences in the target genome (FIG. 4B). The limit of detection of the same probes by the Hyb procedure was 1000-fold lower; and was consistent with the amount of signal amplification with RCA using PCR targets.

The utility of microarray assays for high throughput genotyping is well recognized. However, there is need for a robust genotyping assay that is rapid, cost-effective and scalable. Genotyping assays based on target pre-amplification are hampered by low throughput and high cost. Due to the need for generation of sub-samples of amplified targets prior to genotyping, the total number of assay steps increase enormously. Thus resulting in greater complexity in automation, sample handling, management of genotyping projects and an increased risk of the cross-contamination of amplified products.

This example describes strategies for, on-chip rolling circle amplification (RCA) of genotyping signals generated by single base extension (SBE). SBE was chosen for genotyping on hydrogel microarrays because of the simplicity of the assay as well as the remarkable specificity of DNA polymerases in incorporating modified chain terminating nucleotides. RCA technology was successful in achieving a 3-log increase in the sensitivity of detection of SBE genotyping assays employing SNP-containing amplicon targets. The results suggest that RCA signal amplification may be useful in the improvement of sensitivity of genotyping assays on microarrays, and might also enhance the fidelity of allele discriminating signals. Also, the data indicate that RCA amplification of SBE signals, on hydrogel microarrays, dependably replicates signals generated by SBE and unbiased by target sequences. Signal amplification of genotyping reactions employing genomic targets may afford adequate sensitivity, sample economy, and cost efficiency for genotyping projects. Assay sensitivity needs to be improved for genotyping single copy gene sequences directly from genomic targets. Perhaps, improving hybridization yields using an active hybridization approach, e.g., electronic hybridization, and improving polymerase turnover rates may provide a better SBE yield. Since genome amplification of unit-copy loci is expensive and cumbersome, perhaps approaches to perform pooled amplifications of sets of genetic loci might help to cut costs and complexity in large- scale genotyping projects.

Experimental data herein show that the RCA technology has the required sensitivity for using unmodified human genomic DNA strongly for genotype analysis. Additionally, the compatibility of universal RCA technology with the hydrogel substrates demonstrate the. potential for performing population scale genotyping reactions quickly and efficiently. In turn, the enhanced flow of genotyping information may more quickly lead to a better understanding of the role of DNA sequence variation in human health and disease.

Experimental Protocol

Human genomic SNP targets and polymerase chain reaction (PCR) amplicon preparation. Human genomic DNA was obtained from the Coriell Cell Repositories (DNA Polymorphism Discovery Resource, Cat.#: M08PDR. Sample PD0007 was used exclusively in these protocols; 401 Haddon Ave., Camden, N.J. 08103 ; 800-752-3805). The single nucleotide polymorphic sites (SNP's) and probes used in this study are presented in FIG. 5. All sequence tag sites (STSs) were derived from the dbEST and the Unigene databases.

Polymerase chain reactions (PCR) employed commercial products and reagents (AmpliTaq™, PE Biosystems). In a reaction volume of 100 μl, the final concentrations of reactants were: 50 μM deoxynucleotide triphosphates 0.25 μM for both forward and reverse primers (Operon, Inc.), 100 ng of genomic DNA template, 1×commercial reaction buffer, and 2.5 units of AmpliTaq thermostable DNA polymerase. The amplification procedure employed an MJ Research thermalcycler (PTC-100), and the cycling regimen included an initial denaturation step of 94° C. for 2 minutes, followed by 30 cycles of a three-step amplification regimen of: 94° C., 30 seconds; 60° C., 30 seconds; and 72° C., 1 minute; followed by a final extension step at 72° C. for 5 minutes. The PCR reaction products were electrophoretically examined for yield and purity; with yields determined using a quantitative standard 100 bp DNA ladder, and imaging software (ImageQuant, Molecular Dynamics). Target amplicon preparations were purified using QlAquick PCR Purification Kits (Qiagen, cat.: 28104).

Fragmentation of purified amplicon targets was accomplished by DNasel digestion (Life Technologies, cat.#: 18068015). Each target amplicon was separately digested at a concentration of 10 ng/μl, with 0.02 units/μl of DNasel in vendor-supplied reaction buffer at 37° C. for 10 minutes. The reaction was stopped by incubation at 95° C. for 10 minutes. Nuclease-treated targets were stored at −20° C. until needed for further experimental procedures. Fragmentation of human genomic DNA was performed by Dnasel.

Single nucleotide polymorphism (SNP) chip construction. A set of paired, bi-allelic oligonucleotide probes representing six unit-copy SNP's and other multicopy genomic targets (FIG. 5 and FIG. 4) were synthesized with 5′-amine-(C₆)-linkers (Operon, Inc.). The probes were arrayed onto slides coated with a film of hycdrogel containing activated (NHS) esters (“3D-Link™ slides; cat.#: DN01-0025; Surmodics, Inc.; Eden Prairie, Minn.) employing a modified BioJet II dispense robot (Packard, Meridian, Conn.; Motorola Life Sciences, Tempe, Ariz.). Dispense, blocking reagents and procedures followed the manufacturers specifications.

Oligonucleotides and reagents for single base extension (SBE), immuno-hybridization (Hyb) and immuno-rolling circle amplification (RCA). Oligonucleotide and immuno-conjugate reagents employed in the acquisition and amplification of fluorescent signals generated by means of the SBE reaction included:

Primer 1: 5′- Amine-(C)₁₂(A)₅₀-ACACAGCTGAGGATAGGACATAATAAGC-3′,

Circle 1: 5′-CTC AGC TGT GTA ACA ACA TGA AGA TTG TAG GTC AGA ACT CAC CTG TTA GAA ACT GTG MG ATC GCT TAT TAT GTC CTA TC -3′,

Primer decorator: Primer-Det 1D: 5′-Cy5™-TGT CCT ATC CTC AGC TGG-Cy5-3′,

Circle decorator: Circle-Det1D: 5′-Cy5-CCTACAATCTTCATGTTGTTAC-3′, and α-Biotin IgG-Primer 1 Conjugate (Molecular Staging, Inc.; Custom, 500 ng/μl).

SBE reaction. The single base extension SNP assay employed a DNA polymerase-mediated, 3′ single base extension (SBE) of oligonucleotide probes immobilized onto the surface of hydrogel coated glass slides. The 3′ end of each probe was designed to query annealing target sequences for the ability to mediate the extension of the probe by a single base, using chain-terminating acyclo-nucleoside triphosphates analogs. Probe designations and sequences are presented in FIG. 5.

The slides were placed in custom manufactured, titanium hybridization/reaction chambers (Motorola Life Sciences. Tempe, Ariz.). The 80 μl SBE reactions contained 50 mM Tris-HCI, pH 8.5; 2 mM MgCl₂, 10 mM KCl; 1 μM each of biotinylated acyclo-nucleoside triphosphates (ATP, CTP, GTP, UTP; PerkinElmer Life Sciences, cat.#: CUS 999); 0.2 Units/μl ThermoSequenase DNA polymerase (Amersham Pharmacia Biotech, Cat.#: E79000Y); and 0.1-20 ng DNasel-treated amplicon target. The SBE reaction employed an MJ Research Peltier ThermalCycler, DNA Engine Tetrad (PTC-225), and a thermal cycling regimen with an initial denaturing step of 85° C. for 1 minute, followed by 1 to 20 cycles of a two-step base-extension regimen of 85° C. for 30 seconds, and 60° C. for 10 minutes.

Following the SBE reaction, arrays were rinsed, while still in the reaction chamber, with 100 μl 5×SSC pre-warmed to 60° C. (1×SSC: 150 mM NaCl, 15 mM sodium citrate). The reaction chambers were disassembled and the slides removed to a polypropylene conical. tube (Corning Inc., Corning, N.Y.) containing 45 ml of 60° C., 5×SSCT (5×SSC+0.05% Tween 20, Pierce Chemical Co. cat.: 28320), and incubated for 30 minutes, in a hybridization oven (Lab-line, Model #309) at 60° C., with gentle rotational agitation. The wash buffer was removed and the slides were washed three more times with equal volumes of distilled, de-ionized water (“ddH₂O, >18 Ω) at room temperature for ˜1 minute each, with gentle agitation. A final wash in TE buffer (10 mM Tris-HCL pH 8, 1 mM Na₂EDTA) was performed for 1 hour at room temperature with gentle rotational agitation. These final washing steps were found to decrease non-specific background fluorescence.

Hyb and RCA signal development. SBE-processed slides were dried with a stream of anhydrous, HEPA-filtered nitrogen. Individual arrays were circumscribed with hydrophobic ink (Pap Pen), covered with 80 μl of Blocking Buffer (0.5% Gelatin [Sigma, cat.#: G-2500], 0.5% non-fat dry milk (w/v, Carnation), 1.5% BSA (Sigma, cat.#: B-4287), 5 mM Na2EDTA (Gibco-.BRL), in PBST (phosphate buffered saline [Gibco-BRL, cat.#: 70013-032] containing 0.05% Tween 20 [Pierce Chemical Co., cat.#: 28320]), and incubated at 37° C. for 30 minutes, in a humidity chamber.

For each array, an 80 μl mixture of circle 1 oligonucleotide (50 nM) and α-biotin antibody-primer1 conjugate (1 ng/μl) was incubated (“pre-annealed”) in Blocking Buffer at 37° C. for 30 minutes. After array blocking, the slides were washed twice in PBST, 2 minutes per wash, at room temperature. To each array was added the 80 μl of pre-annealed conjugate, and the slides were incubated at 37° C. for 30 minutes in a standard convection cell incubator (Precision Scientific Model #31534). The slides were washed as before in PBST, with a final wash, at room temperature, for 2 minutes with gentle agitation, in φ29 Reaction Buffer (50 mM Tris-HCI pH 7.9, 10 mM MgCl₂, 10 mM (NH₄)₂SO₄, 2 mg/ml BSA, 0.05% Tween 20).

After removal of excess moisture by spin drying, 80 μl of φ29 buffer, containing 0.2 units/μl of φ29 DNA polymerase, was applied to each array and the slides were incubated for 1 hour at 37° C. (RCA). The same procedure was performed in the absence of polymerase (Hyb). Slides were washed once for 2 min in 2×SSC+0.05% Tween 20, spin dried, and incubated with 0.5 μM decorator oligonucleotide in 2×SSCT) (2×SSC+0.05% Tween 20) for 30 minutes at 37° C. Slides were washed in After washes in 2×SSCT at room temperature, and a final 1 minute wash in 1×SSC, slides were stored in the dark until scanned.

Detection and quantitation of Hyb and RCA signals. Slides were scanned in a GenePix 4000a microarray scanner; and image visualization employed the GenePix™ v. 3.0 software (Axon Instruments). Quantitative data manipulations were performed using CodeLink™ v. 1.4 software (Motorola life Sciences, Temp, Ariz.).

We claim:

1. A method of detecting the presence of a target sequence comprising:
a) providing a device comprising a substrate having a capture probe comprising:
i) a first domain substantially complementary to a open circle probe; and
ii) a second domain comprising a cleavage site;
wherein said capture probe is attached to said substrate at both termini;
b) contacting said capture probe with said target sequence and said open circle probe to form a hybridization complex;
c) contacting said hybridization complex with a ligase such that said open circle probe circularizes to form a second hybridization complex;
d) contacting said capture probe with a cleavage agent to cleave said probe at said cleavage site;
e) adding an extension enzyme and NTPs to said second hybridization complex to form an extended capture probe; and
f) detecting said extended capture probe.

2. A method according to claim 1 wherein said substrate comprises a first electrode to which said capture probe is attached, said device further comprises a second electrode and said detecting comprises measuring impedance between said electrodes.

3. A method according to claim 1 wherein said substrate comprises a first electrode to which said capture probe is attached, and said detecting comprises adding a mediator and detecting the oxidation of guanine.

4. A method according to claim 1 wherein said extended capture probe comprises a label.

5. A method according to claim 4 wherein said label is a fluorescent label.

6. A method according to claim 4 wherein said label is an electron transfer moiety (ETM).

7. A method according to claim 1 wherein said open circle probe comprises a label sequence and said method further comprises hybridizing a label probe comprising a label to said label sequence.

8. A method according to claim 4 wherein said label comprises a hapten and said detecting comprises the addition of a fluorescent binding partner of said hapten.

9. A method of detecting the presence of a target sequence comprising a first and a second target domain, said method comprising:
a) providing a device comprising a substrate comprising a capture probe substantially complementary to a first domain of said target sequence;
b) contacting said capture probe with:
i) said target sequence; and
ii) a rolling circle primer comprising:
1) a first domain substantially complementary to said second domain of said target sequence; and
2) a second domain substantially complementary to a first domain of a circularized probe;
to form a hybridization complex;
c) contacting said hybridization complex with a ligase such that capture probe and said rolling circle primer ligate;
d) hybridizing said second domain of said rolling circle primer to a circularized probe to form a second hybridization complex;
e) adding an extension enzyme and NTPs to said second hybridization complex to form an extended capture probe; and
f) detecting said extended capture probe.

10. A-method according to claim 9 wherein said substrate comprises a first electrode to which said capture probe is attached, said device further comprises a second electrode and said detecting comprises measuring impedance between said electrodes.

11. A method according to claim 9 wherein said substrate comprises a first electrode to which said capture probe is attached, and said detecting comprises adding a mediator and detecting the oxidation of guanine.

12. A method according to claim 9 wherein said extended capture probe comprises a label.

13. A method according to claim 12 wherein said label is a fluorescent label.

14. A method according to claim 12 wherein said label is an electron transfer moiety (ETM).

15. A method according to claim 9 wherein said open circle probe comprises a label sequence and said method further comprises hybridizing a label probe comprising a label to said label sequence.

16. A method according to claim 12 wherein said label comprises a hapten and said detecting comprises the addition of a fluorescent binding partner of said hapten.

17. A method of detecting the presence of a target sequence comprising a first target domain adjacent to a second target domain, said method comprising:
a) providing a device comprising a substrate comprising a capture probe substantially complementary to said second target domain of said target sequence;
b) contacting said capture probe with:
i) said target sequence; and
ii) a ligation probe comprising:
1) a first domain substantially complementary to said second domain of said target sequence; and
2) a rolling circle primer;
wherein when the nucleotides at the adjacent termini of said capture probe and said ligation probe are perfectly complementary to the respective target nucleotides, said capture probe and said ligation probe are ligated to form a ligated probe;
c) hybridizing said rolling circle primer of said ligated probe with a rolling circle priming sequence of a closed circle probe to form a rolling circle hybridization structure;
d) providing an extension enzyme and NTPs such that said ligated probe is extended; and
e) detecting said extended ligated probe.

18. A method according to claim 17 wherein said substrate comprises a first electrode to which said capture probe is attached, said device further comprises a second electrode and said detecting comprises measuring impedance between said electrodes.

19. A method according to claim 17 wherein said substrate comprises a first electrode to which said capture probe is attached, and said detecting comprises adding a mediator and detecting the oxidation of guanine.

20. A method according to claim 17 wherein said extended capture probe comprises a label.

21. A method according to claim 20 wherein said label is a fluorescent label.

22. A method according to claim 20 wherein said label is an electron transfer moiety (ETM).

23. A method according to claim 17 wherein said open circle probe comprises a label sequence and said method further comprises hybridizing a label probe comprising a label to said label sequence.

24. A method according to claim 20 wherein said label comprises a hapten and said detecting comprises the addition of a fluorescent binding partner of said hapten.

25. A method of determining the identification of a nucleotide at a detection position in a target sequence comprising:
a) providing a first hybridization complex comprising said target sequence and a capture probe comprising an interrogation position;
b) contacting said first hybridization complex with an extension enzyme and at least one chain terminating nucleotriphosphate comprising a hapten, under conditions wherein only if the nucleotides at said detection and interrogation positions are perfectly complementary does said capture probe get extended;
c) adding:
i) secondary probe comprising:
1) the binding partner of said hapten; and
2) a rolling circle primer;
ii) closed circle probe comprising a rolling circle priming sequence; to form a second hybridization complex between said rolling circle primer and said rolling circle priming sequence;
d) contacting said second hybridization complex with an extension enzyme and NTPs to extend said primer; and
e) detecting said extended primer.

26. A method according to claim 25, wherein at least one said NTPs comprises said hapten such that a secondary probe will also bind to said hapten on said NTP and form a third hybridization complex, said method further comprising contacting said third hybridization complex with an extension enzyme and NTPs to extend said primer.

27. A method according to claim 25 wherein said substrate-comprises a first electrode to which said capture probe is attached, said device further comprises a second electrode and said detecting comprises measuring impedance between said electrodes.

28. A method according to claim 25 wherein said substrate comprises a first electrode to which said capture probe is attached, and said detecting comprises adding a mediator and detecting the oxidation of guanine.

29. A method according to claim 25 wherein said extended capture probe comprises a label.

30. A method according to claim 29 wherein said label is a fluorescent label.

31. A method according to claim 29 wherein said label is an electron transfer moiety (ETM).

32. A method according to claim 25 wherein said open circle probe comprises a label sequence and said method further comprises hybridizing a label probe comprising a label to said label sequence.

33. A method according to claim 29 wherein said label comprises a hapten and said detecting comprises the addition of a fluorescent binding partner of said hapten.
